(19) United States
(12) Patent Application Publication
(10) Pub. No.: US 2008/0260650 A1
(43) Pub. Date: Oct. 23, 2008

(54) Methods of Detection and Therapy of Inflamed Tissues Using Immune Modulation

(75) Inventors: Ahmed Tawakol, Wayland, MA (US); Michael R. Hamblin, Revere, MA (US); Raymond Q. Migrino, Wauwatosa, WI (US); Jeffrey Gelfand, Cambridge, MA (US)

Correspondence Address: EWARDS ANGELL PALMER & DODGE LLP, P.O. BOX 55874, BOSTON, MA, 02205, US

(73) Assignee: THE GENERAL HOSPITAL Corporation, Boston, MA (US)

(21) Appl. No.: 11/666,573

(22) PCT Filed: Oct. 27, 2005
(86) PCT No.: PCT/US05/38863

§ 371 Date: Mar. 12, 2008

Related U.S. Application Data
(60) Provisional application No. 60/623,032, filed on Oct. 28, 2004.

(51) Int. Cl. A61K 49/06; A61K 38/04; A61P 29/00

(52) U.S. Cl. 424/9.37; 424/9.1; 514/12

(57) ABSTRACT

The present invention relates to methods for the detection and therapy of inflamed tissue, whereby immune modulators are used to increase the uptake of diagnostic or therapeutic compositions by inflammatory cells.

### CROSS-REFERENCE TO RELATED APPLICATIONS/PATENTS & INCORPORATION BY REFERENCE

This application claims priority to U.S. Provisional Application Ser. No. 60/623,032, filed on Oct. 28, 2004, the contents of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List before the paragraphs, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Inflammation is a complex, multifactorial network of interactions among soluble factors and cells that can arise in any tissue in response to traumatic, infectious, post-ischemic, toxic, oncologic or autoimmune injury. The process typically leads to recovery from injury and ultimately, healing of the damaged tissue. However, if targeted destruction and assisted repair are not properly regulated, inflammation can lead to persistent tissue damage by leukocytes, lymphocytes, or collagen. Inflammation may be considered in terms of its checkpoints, whereby binary or higher-order signals drive each commitment to escalate, and molecules responsible for propagating the inflammatory response also suppress it, depending on timing and context (Nathan, C., 2002).

Many novel anti-inflammatory treatment modalities have been designed to modulate the complex process of inflammation. Various strategies have been employed to block crucial steps within this multifactorial process. The vast majority of therapies for inflammation are pharmacological agents. Unfortunately, many pharmacological agents are broad-spectrum, and often have unforeseen, pleiotropic effects, or target one specific aspect of the inflammatory process without taking into consideration that many aspects are redundant and can overcome inhibition of one specific target.

Corticosteroids are often used to reduce inflammation. Corticosteroids cause a decrease in the number of circulating lymphocytes as a result of steroid-induced lysis of lymphocytes, or by alterations in lymphocyte circulation patterns (Kuby, J. (1998) In: Immunology 3^{rd} Edition W.H. Freeman and Company, New York; Pelaia, G. et al. 2003). Corticosteroids affect the regulation of a Rel transcription factor family member, nuclear factor κB (NF-κB) by inducing the upregulation of an inhibitor of NF-κB known as IκB, which sequesters NF-κB in the cytoplasm and prevents it from transactivating pro-inflammatory genes in the nucleus. Corticosteroids also reduce the phagocytic ability of macrophages and neutrophils, as well as reducing chemotaxis. However, the effects of corticosteroids are not specific to the inflammatory response, as they also cause alterations in carbohydrate, protein, and lipid metabolism, and influence processes of the renal, cardiovascular, endocrine, and nervous systems (Goodman Gilman A., Hardman, J. G., Limbird, L. E., Molinoff, P. B., Ruddon, R. W. (eds) Goodman & Gilman's The Pharmacological Basis of Therapeutics 9^{th} Edition. (1996) McGraw-Hill, New York).

Similar effects are observed with the use of inhibitors of NF-κB, TNFα (Keane, J. et al. 2001), and matrix metalloproteinases (Corry, D. B. et al. 2002; Coussens, L. M. et al. 2002). These cellular factors often have seemingly opposing roles in vivo that are regulated by timing and context, thus resulting in unanticipated side effects or lack of efficacy when administered to treat inflammation. While inhibitors to these immunoregulatory molecules can be potentially useful in the future, current therapies are precluded for clinical use.

Monoclonal antibodies against specific receptors involved in leukocyte rolling and adhesion are recent discoveries that may be used to treat inflamed tissues (Boehncke, W. H. et al. 2000). Such antibodies have been directed against mucins sialyl Lewis X, integrins, E, P, and L-selectins, and other adhesion molecules. Other potential targets for monoclonal antibodies include cytokine receptors such as TNFαR, the interleukin receptors, interferon receptors, among others. However, it is important to note that inflammation is a complex network of signals, and the process is governed by redundant mediators and exerted by functionally overlapping molecules and mechanisms. In comparison to corticosteroids and other broad-spectrum inhibitors, which can modulate a wide variety of systems, monoclonal antibodies are highly specific and consequently, can be less effective.

The vast majority of pharmacological agents used to treat inflammation fall into two broad classes: the non-steroidal anti-inflammatory drugs, or NSAIDs, and antihistamines. NSAIDs exert their mechanism of action by blocking eicosanoid biosynthesis. Eicosanoids include prostaglandins, lipooxygenases, leukotrienes, and thromboxanes, which are intimately involved in mediating the inflammatory response. The key enzyme that has been the target of numerous pharmacological studies is the cyclooxygenase (COX) enzyme, of which there are two isoforms. COX-2 is thought to be specific for the inflammatory response (Funk, C. D., 2001). These COX enzymes are directly responsible for the formation of all of the eicosanoids listed above, from a common precursor called arachidonic acid. NSAIDs are well tolerated clinically, however some are known to induce gastric ulceration. However, NSAIDs may act through mechanisms other than inhibition of COX enzyme activity alone, such as inducing apoptosis and caspase activation (Funk, C. D., 2001; Epiriat, J. C. and Gilmore, T. D. 1999). Other related drugs target eicosanoid binding to their cognate receptors, exemplified by cysteinyl leukotriene receptor antagonists. These agents have been primarily used in treatment of vasoconstriction and inflammation associated with asthma (Holgate, S. T. et al. 2003).

Antihistamines form the other broad class of pharmacological agents commonly used to treat inflammation. Antihistamines exert their effects through histamine receptors H1 through H3. Diphenhydramine is a prototypical histamine H1 receptor antagonist (Goodman Gilman A., Hardman, J. G., Limbird, L. E., Molinoff, P. B., Ruddon, R. W. (eds) Goodman & Gilman's The Pharmacological Basis of Therapeutics 9^{th} Edition. (1996) McGraw-Hill, New York). Second generation histamine H1 receptor antagonists also include loratadine, fexofenadine, and other piperidines. Histamine receptors regulate numerous effects in the body, such as smooth muscle relaxation, vasodilation, formation of edema, stimulation of sensory nerve endings, bronchoconstriction, and gastric acid secretion. Many antihistamines have side effects that include sedation, tachycardia, and mutagenicity. Antihistamines are often used for acute allergic responses.

The final class of anti-inflammatory treatments are drugs used to lower cholesterol by impinging on a key enzyme in the cholesterol biosynthetic pathway, 3-hydroxy-3-methylglutaryl-coenzyme A reductase (HMG-CoA reductase) (reviewed in Weitz-Schmidt, G. 2002). These drugs are collectively known as statins. Recent clinical evidence indicates that while statins reduce cardiovascular-related morbidity and mortality, they also impact leukocyte migration (Diomede, L. et al. 2001). Downregulation of the cytokines MCP-1, IL-6, and the chemokine RANTES were observed, as well as downregulation of endothelial and leukocyte adhesion molecules (Yoshida, M. et al. 2001; Romano, M. et al. 2000). Additionally, statins are believed to cause downregulation of adhesion molecule expression and cytokine and chemokine release (Niwa, S. et al. 1996).

Current therapies treat the result of inflammation, not the cause and as a result, suffer from either a lack of specificity or breadth in providing a therapeutic effect. There exists a need in the art for therapies that extensively target the source of inflammation, which would involve the regulation of inflammatory cells at the site of injury.

### SUMMARY OF THE INVENTION

It has now been shown that modulators of the immune system can increase the selective targeting of diagnostic and therapeutic compositions to a site of inflammation. Methods of the present invention employ immune modulators to increase the uptake of diagnostic or therapeutic compositions by inflammatory cells associated with inflamed tissue, thereby improving diagnosis and therapy.

One aspect of the present invention provides a method of identifying inflamed tissue in a subject, the method comprising the steps of:
(a) administering a diagnostic composition;
(b) administering an immune modulator that increases localization of the diagnostic composition to inflammatory cells of the inflamed tissue;
(c) detecting the diagnostic composition; and
(d) identifying the inflamed tissue in the subject.

The diagnostic composition can be comprised of a diagnostic agent coupled to a molecular carrier. In some embodiments, the diagnostic agent is internalized by the inflammatory cells.

Diagnostic agents can be but are not limited to photosensitizers, radiolabeled markers (e.g., radionuclides, paramagnetic contrast agents, β-emitters) and fluorescent markers.

Diagnostic methods of the present invention can comprise further steps, wherein an inflamed tissue, such as an infected tissue, is identified, and distinguished from other tissue, including tumors. Accordingly, in another aspect, the present invention provides a method for identifying inflamed tissue in a subject, the method comprising the steps of:
a) administering a diagnostic composition;
b) administering an immune modulator that increases localization of the diagnostic composition to inflammatory cells of the inflamed tissue;
c) comparing a signal emitted by the diagnostic composition in the inflamed tissue to a signal emitted by the diagnostic composition in another tissue; and
d) determining the location of the greater amount of signal to thereby identify the inflamed tissue in the subject.

In yet another aspect, the present invention provides a method for treating inflamed tissue in a subject in need thereof, the method comprising the steps of:
a) administering a therapeutic composition;
b) administering an immune modulator that increases localization of the therapeutic composition to inflammatory cells of the inflamed tissue, thereby treating the subject for inflamed tissue.

The therapeutic composition can be comprised of a therapeutic agent coupled to a molecular carrier. In some embodiments, the therapeutic agent is internalized by the inflammatory cells.

Molecular carriers can be but are not limited to serum proteins, receptor ligands, microspheres, liposomes, antibodies, growth factors, peptides, hormones and lipoproteins. In specific embodiments, the molecular carriers are targeted to scavenger receptors, T-cells, or macrophages.

The immune modulator can be but is not limited to a colony stimulating factor, interleukin, interferon, chemokines, chemoattractant, growth factor, inhibitory factor, bacterially derived epitope or signal transduction molecule.

In specific embodiments, the immune modulator is GM-CSF, M-CSF, G-CSF, interleukins-1 through 29 (abbreviated IL-1, IL-2, and so on), TNFαc, formyl-methionineleucine-phenylalanine (fMLP), endotoxins, and lipopolysaccharide (LPS), phorbol 12-myristate-13-acetate, interferon α, interferon β, interferon γ, CD40, ligands of CD40 (e.g., gp39), MCP-1 through 5, bFGF, muramyl dipeptide, urokinase, a C—, CC—, CXC— or CX3C family member, RANTES, GRO α, β, γ, I-TAC, MIG-1, LIF, oncostatin M, TGF β, TIMP, MCF, and MIP-1 through 5, or α, β, δ, γ isoforms thereof.

The inflammatory cells in which diagnostic and therapeutic compositions of the invention are localized, and optionally internalized, include but are not limited to smooth muscle cells, dendritic cells, follicular dendritic cells, Langerhans cells, interstitial, interdigitating, blood, and veiled dendritic cells, leukocytes, natural killer cells, lymphocytes, monocytes, macrophages, alveolar macrophages, microglia, mesangial cells, histiocytes, Kupffer cells, foam cells, mast cells, endothelial cells, megakaryocytes, platelets, erythrocytes and polymorphonuclear cells.

Diseases and conditions for which subjects of the present invention may undergo treatment or diagnosis include but are not limited to acute or chronic infectious disorders (including bacterial, viral, and prion diseases) and autoimmune disorders (such as systemic lupus, arthridites, vasculidites).

In yet another aspect, the present invention provides a method for identifying and treating inflamed tissue in a subject in need thereof with the use of photodynamic means, the method comprising the steps of:
a) administering at least one photosensitizer;
b) administering an immune modulator that increases localization of the photosensitizer to inflammatory cells of the inflamed tissue;
c) detecting a sufficient amount of the photosensitizer to thereby identify the inflamed tissue; and
d) irradiating the photosensitizer to produce a phototoxic species that destroys the inflammatory cells, thereby treating the subject for inflamed tissue.

In yet another aspect, the present invention provides a kit for identifying inflamed tissue comprising a diagnostic agent, an immune modulator and instructions for using the diagnostic agent and the immune modulator to identify inflamed tissue in accordance with the methods of the invention.

In one embodiment, the kit of includes a detector for detecting the diagnostic agent.

In yet another aspect, the present invention provides a kit for treating inflamed tissue in a subject in need thereof comprising a therapeutic agent, an immune modulator and instructions for treating inflamed tissue using the therapeutic agent and the immune modulator in the subject in accordance with the methods of the invention.

In yet another aspect, the present invention provides a kit for detecting and treating inflamed tissue in a subject in need thereof comprising a photosensitizer, an immune modulator, and instructions for using the photosensitizer and the immune modulator to detect and treat inflamed tissue in a subject in accordance with the methods of the invention.

Other aspects of the invention are described in or are obvious from the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1 shows positron emission tomography (PET) images of Candida-inoculated mice before and after GMCSF administration.

### DETAILED DESCRIPTION OF THE INVENTION

### I. Definitions

“Inflammatory cells” are cells that contribute to an immune response, and can include but are not limited to smooth muscle cells, dendritic cells, follicular dendritic cells, Langerhans cells, interstitial, interdigitating, blood, and veiled dendritic cells, leukocytes, natural killer cells, lymphocytes (B-lymphocytes and T-lymphocytes), monocytes, macrophages, foam cells, tissuespecific macrophages such as alveolar macrophages, microglia, mesangial cells, histiocytes, and Kupffer cells, mast cells, endothelial cells, megakaryocytes, platelets, erythrocytes and polymorphonuclear cells (e.g., granulocytes such as basophils, eosinophils, neutrophils).

The term “inflamed tissue” is used to describe any biological tissue in which an adverse immune response to a stimulus is mounted. A stimulus can be, for example, bacterial, fungal, viral, prion and other infectious agents. The adverse immune response can also arise from transplantation or disease, such as autoimmune disease.

The term “immune modulator” refers to any molecule capable of activating an inflammatory cell. “Activation” of inflammatory cells is a phenomenon well known in the art, involving an increase in metabolic and signaling activity by inflammatory cells in response to a stimulus. One manifestation of activation is an increase in ligand uptake and receptor turnover. Other manifestations include changes in cell size, mobility, complexity and proliferative capacity, as well as permanent or transient changes in gene expression. Activated inflammatory cells have increased cell surface binding and internalization capacity. Thus, by way of activation, immune modulators can “localize” such diagnostic and therapeutic agents of the invention to inflammatory cells.

A “molecular carrier” refers to a biomolecule with targeting specificity for inflamed tissues. Molecular carriers are delivery vehicles that “target” therapeutic or diagnostic agents of the invention to inflammatory cells or other inflammatory components for which they have affinity.

As used herein, a “β-emitter” is a composition, such as a radionuclide or a paramagnetic contrast agent, that emits electron or positron rays (“β rays”).

The term “obtaining” as in “obtaining the diagnostic agent” is intended to include purchasing, synthesizing or otherwise acquiring the diagnostic agent (or indicated substance or material).

The term “photosensitizer” refers to a photoactivatable compound, or a biological precursor thereof, that produces a reactive species (e.g., oxygen) having a photochemical (e.g., cross linking) or phototoxic effect on a cell, cellular component or biomolecule.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

### II. Methods and Compositions of the Invention

The present invention provides methods for detecting and/or treating inflamed tissue by co-administering a diagnostic and/or therapeutic composition with an immune modulator, whereby the immune modulator stimulates the localization, and optionally internalization, of the composition by inflammatory cells associated with the inflamed tissue. In this way, methods of the present invention enable the enhanced uptake of diagnostic and/or therapeutic compositions by inflamed tissues, thus enhancing their detection and treatment. Use of the immune modulators to stimulate uptake minimizes the effect of non-specific uptake by other tissues. This decrease in the “signal-to-noise” ratio increases the specificity of detection and treatment.

A therapeutic composition according to the invention can contain a suitable concentration of an active agent (referred to herein as a therapeutic agent) and may also comprise certain other components. For example, in some embodiments, therapeutic compositions of the present invention are formulated with pharmaceutically acceptable carriers or excipients, such as water, saline, aqueous dextrose, glycerol, or ethanol, and may also contain auxiliary substances such as wetting or emulsifying agents, and pH buffering agents in addition to the therapeutic agent. The therapeutic composition can also be comprised of a therapeutic agent coupled to a molecular carrier that has an affinity for inflammatory cells or inflammatory components.

Therapeutic agents can be any treatment modality known in the art for the treatment of inflammation, including but not limited to acute or chronic infectious disorders (including bacterial, viral, and prion diseases) and autoimmune disorders (such as systemic lupus, arthridites, vasculidites).

Methods of the present invention can be used to improve efficacy of surgical and drug treatments that target inflamed tissues. For example, diagnostic and/or therapeutic methods of the invention can be carried out together with treatment schemes known in the art. Several such treatment schemes are contemplated, including anti-infectious agents (e.g., antibiotic therapy, antiviral therapy), and immunosuppression with immunosuppressive agents.

An anti-infectious agent as used herein is an agent which reduces the activity of or kills a microorganism and includes but is not limited to Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alam ecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacir; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparocle; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindainycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oxinonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride.

In certain embodiments, therapeutic agents are immunosuppressants. Such immunosuppressants include but are not limited to Azathioprine; Azathioprine Sodium; Cyclosporine; Daltroban; Gusperimus Trihydrochloride; Sirolimus; Tacrolimus; Everolimus; Actinomycin D; Paclitaxel; Hydroxychloroquine; Adrenocorticosteroids; Cyclosporin; Tacrolimus (FK506); Sulfasalazine; Methoxsalen; Methotrexate; Mycophenolic acid (mycophenolate mofetil); Azathiprine; and NOX-100.

In other embodiments, the therapeutic agents are anti-inflammatory agents. Such anti-inflammatory agents include but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; FIufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; and Zomepirac Sodium.

Administration regimens of these and other therapeutic agents are known in the art and are described, for example by “immunosuppressive Agents” in Goodman & Gilman's “The pharmacological basis of therapeutics” 9th Ed. (Hardman et al. eds) McGraw-Hill pp. 1264-1275 and the Physician's Desk Reference 58^{th} Ed., Thompson.

The present invention further provides methods to identify inflamed tissues by targeting diagnostic compositions to inflammatory cells of the tissue and employing one or more additional means to identify the diagnostic composition. Like therapeutic compositions of the invention, diagnostic compositions can comprise a suitable concentration of a diagnostic agent and can also comprise certain other components such as pharmaceutically acceptable carriers or excipients, such as water, saline, aqueous dextrose, glycerol, or ethanol, and may also contain auxiliary substances such as wetting or emulsifying agents, and pH buffering agents. The diagnostic composition can also be comprised of a diagnostic agent coupled to a molecular carrier. Diagnostic compositions of the invention emit signals that can be detected by standard means known in the art, including but not limited to thermal detection, intravascular ultrasound, intravascular thermography, Raman spectroscopy, angioscopy, near-infrared spectroscopy, intravascular nuclear probes, intravascular electrical impedance imaging, elastography, optical coherence tomography, magnetic resonance imaging, positron emission tomography, single photon emission computed tomography, or other detection modalities known in the art.

A. Characterization of the Immune Response

A stimulus or “trigger” of an immune response can involve at least three different types of signals. First, in response to pain, neurons release bioactive peptides that trigger an immune response. Second, damaged or injured cells release constitutively expressed intracellular proteins that trigger production of soluble immunoregulatory molecules known as cytokines. These intracellular proteins include heat shock proteins, and bacterially derived peptides such as formyl-Methionine-Lysine-Proline (fMLP). Third, microbes and their secreted products are detected by host cells through binding of their conserved molecular constituents to soluble receptors such as complement, mannose-binding protein, lipopolysaccharide (LPS)-binding protein, and to cell-surface receptors such as Toll family members, peptidoglycan recognition proteins, and scavenger receptors (Li, Q. and Verma, I. M., 2002).

These signals ultimately result in the release of histamine, eicosanoids, tumor necrosis factor (TNF), newly synthesized cytokines, tryptases, other proteases, and chemokines from perivascular mast cells. Histamines, eicosanoids and tryptases cause vasodilation and extravasation of fluid. Mast cell tryptases cleave protease-activated receptors that then interact with G-protein coupled receptors on mast cells, sensory nerve endings, endothelial cells, and neutrophils (Lee, D. M. et al., 2002). These actions further activate mast cells and neurons, increase the adhesion of the endothelium to leukocytes via cell adhesion molecules VCAM-1, MadCAM-1 and the E, L, and P-selectins, while simultaneously increasing the “leakiness” of the endothelial wall, and finally, prompt leukocytes to release platelet activating factor (PAF). PAF reinforces the pro-adhesive conversion of the endothelium, which results in leukocyte emigration from the vasculature, which is also known as “extravasation.”

Neutrophils are partially activated by TNF and other factors produced by mast cells and other neutrophils, such as leukotrienes. This leads to release of small amounts of elastase, which cleaves the anti-adhesive barrier known as leukosialin from neutrophil cell surfaces (Nathan, C., et al., 1993). The cleavage event ultimately results in the binding of these newly exposed integrin receptors to extracellular matrix proteins. The binary signal of integrin engagement plus TNF-, chemokine-, or complement-mediated stimulation triggers massive degranulation of the neutrophil, and a phenomenon known as “respiratory burst” (Nathan, C. F. 1987). The components of this respiratory burst include proteases, hydrolases, bacterial permeability increasing factor, α-defensins, serprocidins, azurocidin, and factors that promote formation of reactive oxygen species (ROS), like hydrogen peroxide, hypohalites, and chloramines. The oxidants activate matrix metalloproteinases (“MMPs”) and inactivate protease inhibitors (Weiss, S. J., 1989).

MMPs cleave TNF from tissue macrophages as well as from monocytes that are chemotactically attracted from the bloodstream into tissues by azurocidin (Morgan, J. G., et al., 1991). Macrophage and monocyte derived TNF and chemokines attract and activate more neutrophils, and also combined with mast cell-derived prostaglandin E2 (PGE2) and neutrophilderived defensins to recruit lymphocytes (Yang, D., 1999). Leukotrienes also present in the microenvironment help to attract antigen-presenting dendritic cells (Robbiani, D. F., et al. 2000). In combination with antigen presentation, lymphocytes activate macrophages to secrete proteases, eicosanoids, cytokines, ROS and reactive nitrogen species (RNS). Each component of the immune response is optimized for acceleration, but requires ongoing verification to avoid defaulting to the resting state.

Chemokines, which are soluble factors responsible for recruitment of inflammatory cells, are also used by tumor cells to further their growth and progression. The GRO (growth regulated oncogene) family of cytokines exerts autocrine control over neoplastic cell proliferation (Richmond, A., and Thomas, H. 1986). Tumor growth is also encouraged by the proliferation of new blood vessels through a process called angiogenesis. Many chemokines of the CXC subfamily are pro-angiogenic and stimulate endothelial cell chemotaxis (Vicari, A. P. and Caux, C. 2002). Malignant cells that possess metastatic capacity have properties endowing them with the ability to survive in ectopic tissue, and one way of achieving this migration is through the CXCL12 chemokine, which has been shown to trigger chemotaxis of a variety of malignant tumors in vitro (Muller, A. et al. 2001).

Tumor cells not only take advantage of the trophic factors produced by inflammatory cells, but also use the same adhesion molecules to aid in migration and horning during metastatic spread. Selectins, or adhesion receptors, normally recognize certain vascular mucin-type glycoproteins that bear the structure sialyl-Lewis X and facilitate leukocyte rolling along the blood vessel wall. Metastatic progression of many carcinomas correlates with tumor production of mucins containing sialyl-Lewis X (Zhang, J. et al. 2002). Lung colonization by melanoma cells that express sialyl-Lewis X is significantly reduced in mice that are deficient in E and P-selectins (Kim, Y. J. et al. 1998). P-selectin deficiency attenuates tumor growth and metastasis, and tumors are significantly smaller in mice treated with a receptor antagonist peptide. In summary, neoplastic cells commandeer the same endogenous machinery used by the body to protect itself from injury and microbial invasion to further its own uncontrolled growth and expansion.

B. Immune Modulators

Immune modulators of the present invention encompass diverse categories and sub-categories of molecules known in the art to activate inflammatory cells, including the colony stimulating factors, the interleukins, the interferons, the chemokines/chemoattractants, growth factors, inhibitory factors, peptides and bacterially derived epitopes, and signal transduction molecules. Immune modulators can be soluble or membrane-bound and can consist, for example, not only of receptors, but also of the ligands for receptors.

The colony stimulating factors include but are not limited to granulocyte-macrophage colony stimulating factor (GM-CSF), macrophage colony stimulating factor (M-CSF) and granulocyte colony stimulating factor (G-CSF).

The interleukins include but are not limited to interleukins-1 through 29 (abbreviated IL-1, IL-2, and so on).

The interferons include but are not limited to interferon α, interferon β, interferon γ, isoforms and splice variants thereof.

The chemokines and chemoattractants include but are not limited to categories and subcategories of the C—, CC—, CXC-, and CX3C family members, RANTES (regulated upon activation normal T-cell expressed and presumably secreted), interferon-inducible T-cell alpha chemoattractant (1-TAC), monocyte chemoattractant protein-1 through 5 (MCP-1 through 5) and macrophage chemotactic factor (MCF).

The growth factors include but are not limited to growth regulated oncogenes (GRO) α, β, γ, basic fibroblast growth factor (bFGF) and transforming growth factor β (TGF β).

The inhibitory factors include but are not limited to tissue inhibitors of metalloproteinases (TIMP), leukemia inhibitory factor (LIF), Membrane inhibitor of reactive lysis (MIRL), anaphylatoxin inactivator, C1 inhibitor (C1Inh) and oncostatin M.

The peptides and bacterially derived epitopes include but are not limited to formylmethionine-leucine-phenylalanine (fMLP), endotoxins, muramyl dipeptide and lipopolysaccharide (LPS).

The signal transduction molecules include but are not limited to tumor necrosis factor α (TNFα), phorbol esters such as phorbol 12-myristate-13-acetate (PMA), CD40, ligands of CD40 such as gp39, urokinase, prolactin (PRL), monokine induced by gamma-interferon (MIG-1), macrophage inflammatory protein-1 through 5 including isoforms α, β, δ, γ (MIP-1 through 5), opsonins, complement proteins C1 through C9 as well as any products of proteolysis, regulators of complement proteins such as Factor B, Factor D, Factor H, Factor I, properdin, C4b-binding protein, Membrane-cofactor protein (MCB), Decay-accelerating factor (DAF), S protein and Homologous restriction factor (HRF).

Several of the immune modulators described above are FDA approved and commercially available. FDA approved interferons include interferon alfa-2a (Roferon-A®, Hoffmann-La Roche, Inc.), peginterferon alfa-a (Pegasys®, Hoffmann-La Roche, Inc.), interferon alfa-2b (Intron A®, Schering-Plough Corporation), PEGylated interferon alfa-2b (PEG-Intron™, Schering-Plough Corporation), interferon alfa-n1 (Wellferon®, GlaxoSmithKline), interferon alfa-n3 (Alferon N®, Interferon Sciences, Inc.), interferon beta-1a (Avonex®, Biogen, Inc.; and Rebif®, Serono, Inc.), interferon beta-1b (Betaseron®, Chiron Corp. and Berlex Laboratories), interferon gamma-1b (Actimmune®, Intermune Pharmaceuticals, Inc.). In addition, GM-CSF has been approved by the FDA under the tradename of Leukine® (Berlex Laboratories) and IL-2 has been approved for use under the tradename Proleukin® (Chiron Corp®).

C. Photosensitizer Compositions

Photosensitizers known in the art are typically selected for use according to: 1) efficacy in delivery, 2) proper localization in target tissues, 3) wavelengths of absorbance, 4) proper excitatory wavelength, 5) purity, and 6) in vivo effects on pharmacokinetics, metabolism, and reduced toxicity.

A photosensitizers for clinical use is optimally amphiphilic, meaning that it shares the opposing properties of being water-soluble, yet hydrophobic. The photosensitizer should be water-soluble in order to pass through the bloodstream systemically, however it should also be hydrophobic enough to pass across cell membranes. Modifications, such as attaching polar residues (amino acids, sugars, and nucleosides) to the hydrophobic porphyrin ring, can alter polarity and partition coefficients to desired levels. Such methods of modification are well known in the art.

Without being bound by theory, it is believed that photosensitizers of the present invention can bind to lipoproteins present in the bloodstream and be transported to inflammatory cells located at the site of inflammation. Uptake by inflammatory cells is expedited with the aid of immune modulators. As a result, photosensitizers are selectively delivered to these cells at a higher level and with faster kinetics.

In specific embodiments, photosensitizers of the present invention absorb light at a relatively long wavelength, thereby absorbing at low energy. Low-energy light can travel further through tissue than high-energy light, which becomes scattered. Optimal tissue penetration by light occurs between about 650 and about 800 nm. Porphyrins found in red blood cells typically absorb at about 630 nm, and new, modified porphyrins have optical spectra that have been “red-shifted”, in other words, absorbs lower energy light. Other naturally occurring compounds have optical spectra that is red-shifted with respect to porphyrin, such as chlorins found in chlorophyll (about 640 to about 670 nm) or bacteriochlorins found in photosynthetic bacteria (about 750 to about 820 nm).

Photosensitizers of the invention can be any known in the art, and optionally coupled to molecular carriers.

i) Porphyrins and Hydroporphyrins

Porphyrins and hydroporphyrins can include, but are not limited to, Photofrin® (porfimer sodium), hematoporphyrin IX, hematoporphyrin esters, dihematoporphyrin ester, synthetic diporphyrins, O-substituted tetraphenyl porphyrins (picket fence porphyrins), 3,1-meso tetrakis (o-propionamido phenyl) porphyrin, hydroporphyrins, benzoporphyrin derivatives, benzoporphyrin monoacid derivatives (BPD-MA), monoacid ring “a” derivatives, tetracyanoethylene adducts of benzoporphyrin, dimethyl acetylenedicarboxylate adducts of benzoporphyrin, endogenous metabolic precursors, δ-aminolevulinic acid, benzonaphthoporphyrazines, naturally occurring porphyrins, ALA-induced protoporphyrin IX, synthetic dichlorins, bacteriochlorins of the tetra(hydroxyphenyl) porphyrin series, purpurins, tin and zinc derivatives of octaethylpurpurin, etiopurpurin, tin-etio-purpurin, porphycenes, chlorins, chlorin e₆, mono-1-aspartyl derivative of chlorin e₆, di-1-aspartyl derivative of chlorin e₆, tin(IV) chlorin e₆, meta-tetrahydroxyphenylchlorin, chlorin e₆ monoethylendiamine monamide, verdins such as, but not limited to zinc methylpyroverdin (ZNMPV), copro II verdin trimethyl ester (CVTME) and deuteroverdin methyl ester (DVME), pheophorbide derivatives, and pyropheophorbide compounds, texaphyrins with or without substituted lanthanides or metals, lutetium (III) texaphyrin, and gadolinium(III) texaphyrin.

Porphyrins, hydroporphyrins, benzoporphyrins, and derivatives are all related in structure to hematoporphyrin, a molecule that is a biosynthetic precursor of heme, which is the primary constituent of hemoglobin, found in erythrocytes. First-generation and naturally occurring porphyrins are excited at about 630 nm and have an overall low fluorescent quantum yield and low efficiency in generating reactive oxygen species. Light at about 630 nm can only penetrate tissues to a depth of about 3 mm, however there are derivatives that have been ‘red-shifted’ to absorb at longer wavelengths, such as the benzoporphyrins BPD-MA (Verteporfin). Thus, these ‘red-shifted’ derivatives show less collateral toxicity compared to first-generation porphyrins.

Chlorins and bacteriochlorins are also porphyrin derivatives, however these have the unique property of hydrogenated exo-pyrrole double bonds on the porphyrin ring backbone, allowing for absorption at wavelengths greater than about 650 nm. Chlorins are derived from chlorophyll, and modified chlorins such as meta-tetrahydroxyphenylchlorin (mTHPC) have functional groups to increase solubility. Bacteriochlorins are derived from photosynthetic bacteria and are further red-shifted to about 740 nm. A specific embodiment of the invention uses chlorinₑ₆.

Purpurins, porphycenes, and verdins are also porphyrin derivatives that have efficacies similar to or exceeding hematoporphyrin. Purpurins contain the basic porphyrin macrocycle, but are red-shifted to about 715 nm. Porphycenes have similar activation wavelengths to hematoporphyrin (about 635 nm), but have higher fluorescence quantum yields. Verdins contain a cyclohexanone ring fused to one of the pyrroles of the porphyrin ring. Phorbides and pheophorbides are derived from chlorophylls and have 20 times the effectiveness of hematoporphyrin. Texaphyrins are new metal-coordinating expanded porphyrins. The unique feature of texaphyrins is the presence of five, instead of four, coordinating nitrogens within the pyrrole rings. This allows for coordination of larger metal cations, such as trivalent lanthanides. Gadolinium and lutetium are used as the coordinating metals. In a specific embodiment, the photosensitizer can be Antrin®, otherwise known as motexafin lutetium.

5-aminolevulinic acid (ALA) is a precursor in the heme biosynthetic pathway, and exogenous administration of this compound causes a shift in equilibrium of downstream reactions in the pathway. In other words, the formation of the immediate precursor to heme, protoporphyrin IX, is dependent on the rate of 5-aminolevulinic acid synthesis, governed in a negative-feedback manner by concentration of free heme. Conversion of protoporphyrin IX is slow, and where desired, administration of exogenous ALA can bypass the negative-feedback mechanism and result in accumulation of phototoxic levels of ALA-induced protoporphyrin IX. ALA is rapidly cleared from the body, but like hematoporphyrin, has an absorption wavelength of about 630 nm.

First-generation photosensitizers are exemplified by the porphyrin derivative Photofrin®, also known as porfimer sodium. Photofrin® is derived from hematoporphyrin-IX by acid treatment and has been approved by the Food and Drug Administration for use in PDT. Photofrin® is characterized as a complex and inseparable mixture of monomers, dimers, and higher oligomers. There has been substantial effort in the field to develop pure substances that can be used as successful photosensitizers. Thus, in a specific embodiment, the photosensitizer is a benzoporphyrin derivative (“BPD”), such as BPD-MA, also commercially known as Verteporfin. U.S. Pat. No. 4,883,790 describes BPDs. Verteporfin has been thoroughly characterized (Richter et al., 1987; Aveline et al., 1994; Levy, 1994) and it has been found to be a highly potent photosensitizer for PDT. Verteporfin has been used in PDT treatment of certain types of macular degeneration, and is thought to specifically target sites of new blood vessel growth, or angiogenesis, such as those observed in “wet” macular degeneration. Verteporfin is typically administered intravenously, with an optimal incubation time range from 1.5 to 6 hours. Verteporfin absorbs at 690 nm, and is activated with commonly available light sources. One tetrapyrrole-based photosensitizer having recent success in the clinic is MV0633 (Miravant). MV0633 is well suited for cardiovascular therapies and as such, can be used in therapeutic and diagnostic methods of the invention.

In specific embodiments, the photosensitizer has a chemical structure that includes multiple conjugated rings that allow for light absorption and photoactivation, e.g., the photosensitizer can produce singlet oxygen upon absorption of electromagnetic irradiation at the proper energy level and wavelength. Such specific embodiments include motexafin lutetium (Antrin®) and chlorinₑ₆.

ii) Cyanine and Other Photoactive Dyes

Cyanine and other dyes include but are not limited to merocyanines, phthalocyanines with or without metal substituents, chloroaluminum phthalocyanine with or without varying substituents, sulfonated aluminum PC, ring-substituted cationic PC, sulfonated AlPc, disulfonated and tetrasulfonated derivative, sulfonated aluminum naphthalocyanines, naphthalocyanines with or without metal substituents and with or without varying substituents, tetracyanoethylene adducts, nile blue, crystal violet, azure β chloride, rose bengal, benzophenothiazinium compounds and phenothiazine derivatives including methylene blue.

Cyanines are deep blue or purple compounds that axe similar in structure to porphyrins. However, these dyes are much more stable to heat, light, and strong acids and bases than porphyrin molecules. Cyanines, phthalocyanines, and naphthalocyanines are chemically pure compounds that absorb light of longer wavelengths than hematoporphyrin derivatives with absorption maxima at about 680 nm. Phthalocyanines, belonging to a new generation of substances for PDT are chelated with a variety of diamagnetic metals, chiefly aluminum and zinc, which enhance their phototoxicity. A ring substitution of the phthalocyanines with sulfonated groups will increase solubility and affect the cellular uptake. Less sulfonated compounds, which are more lipophilic, show the best membrane-penetrating properties and highest biological activity. The kinetics are much more rapid than those of HPD, where, for example, high tumor to tissue ratios (8:1) were observed after 1-3 hours. The cyanines are eliminated rapidly and almost no fluorescence can be seen in the tissue of interest after 24 hours.

Other photoactive dyes such as methylene blue and rose bengal, are also used for photodynamic therapy. Methylene blue is a phenothiazine cationic dye that is exemplified by its ability to specifically target mitochondrial membrane potential. Rose-bengal and fluorescein are xanthene dyes that are well documented in the art for use in photodynamic therapy. Rose bengal diacetate is an efficient, cell-permeant generator of singlet oxygen. It is an iodinated xanthene derivative that has been chemically modified by the introduction of acetate groups. These modifications inactivate both its fluorescence and photosensitization properties, while increasing its ability to cross cell membranes. Once inside the cell, esterases remove the acetate groups and restore rose bengal to its native structure. This intracellular localization allows rose bengal diacetate to be a very effective photosensitizer.

iii) Other Photosensitizers

Diels-Alder adducts, dimethyl acetylene dicarboxylate adducts, anthracenediones, anthrapyrazoles, aminoanthraquinone, phenoxazine dyes, chalcogenapyrylium dyes such as cationic selena and tellurapyrylium derivatives, cationic imminium salts, and tetracyclines are other compounds that also exhibit photoactive properties and can be used advantageously in photodynamic therapy. Other photosensitizers that do not fall in either of the aforementioned categories have other uses besides photodynamic therapy, but are also photoactive. For example, anthracenediones, anthrapyrazoles, aminoanthraquinone compounds are often used as anticancer therapies (i.e. mitoxantrone, doxorubicin). Chalcogenapyrylium dyes such as cationic selena- and tellurapyrylium derivatives have also been found to exhibit photoactive properties in the range of about 600 to about 900 nm range, more preferably from about 775 to about 850 nm. In addition, antibiotics such as tetracyclines and fluoroquinolone compounds have demonstrated photoactive properties.

iv) Devices and Methods for Photoactivation

Typically, administration of photosensitizers is followed by a sufficient period of time to allow accumulation of the photosensitizer at the target site. Following this period of time, the photosensitizer is activated by irradiation. This is accomplished by applying light of a suitable wavelength and intensity, for an effective length of time, at the site of the inflammation. As used herein, “irradiation” refers to the use of light to induced a chemical reaction of a photosensitizer.

The suitable wavelength, or range of wavelengths, will depend on the particular photosensitizer(s) used, and can range from about 450 nm to about 550 nm, from about 550 nm to about 650 nm, from about 650 nm to about 750 nm, from about 750 nm to about 850 nm and from about 850 nm to about 950 nm.

In specific embodiments, target tissues are illuminated with red light. Given that red and/or near infrared light best penetrates mammalian tissues, photosensitizers with strong absorbances in the range of about 600 nm to about 900 nm are optimal for PDT. For photoactivation, the wavelength of light is matched to the electronic absorption spectrum of the photosensitizer so that the photosensitizer absorbs photons and the desired photochemistry can occur. Wavelength specificity for photoactivation generally depends on the molecular structure of the photosensitizer. Photoactivation can also occur with sub-ablative light doses. Determination of suitable wavelength, light intensity, and duration of illumination is within ordinary skill in the art.

The effective penetration depth, δ_{eff}, of a given wavelength of light is a function of the optical properties of the tissue, such as absorption and scatter. The fluence (light dose) in a tissue is related to the depth, d, as: e^{-d}/δ_{eff}. Typically, the effective penetration depth is about 2 to 3 mm at 630 nm and increases to about 5 to 6 nm at longer wavelengths (about 700 to about 800 nm) (Svaasand and Ellingsen, (1983) Photochem Photobiol. 38:293-299). Altering the biologic interactions and physical characteristics of the photosensitizer can alter these values. In general, photosensitizers with longer absorbing wavelengths and higher molar absorption coefficients at these wavelengths are more effective photodynamic agents.

Photoactivating dosages depend on various factors, including the amount of the photosensitizer administered, the wavelength of the photoactivating light, the intensity of the photoactivating light, and the duration of illumination by the photoactivating light. Thus, the dose can be adjusted to a therapeutically effective dose by adjusting one or more of these factors. Such adjustments are within the level of ordinary skill in the art.

The light for photoactivation can be produced and delivered to the site of inflammation by any suitable means known in the art. Photoactivating light can be delivered to the site of inflammation from a light source, such as a laser or optical fiber. Preferably, optical fiber devices that directly illuminate the site of inflammation deliver the photoactivating light. For example, the light can be delivered by optical fibers threaded through small gauge hypodermic needles. Light can be delivered by an appropriate intravascular catheter, such as those described in U.S. Pat. Nos. 6,246,901 and 6,096,289, which can contain an optical fiber. Optical fibers can also be passed through arthroscopes. In addition, light can be transmitted by percutaneous instrumentation using optical fibers or cannulated waveguides. For open surgical sites, suitable light sources include broadband conventional light sources, broad arrays of lightemitting diodes (LEDs), and defocused laser beams.

Delivery can be by all methods known in the art, including transillumination. Some photosensitizers can be activated by near infrared light, which penetrates more deeply into biological tissue than other wavelengths. Thus, near infrared light is advantageous for transillumination. Transillumination can be performed using a variety of devices. The devices can utilize laser or non-laser sources, (e.g., lightboxes or convergent light beams).

Where treatment is desired, the dosage of photosensitizer composition, and light activating the photosensitizer composition, is administered in an amount sufficient to produce a phototoxic species. For example, where the photosensitizer is chlorinₑ₆, administration to humans is in a dosage range of about 0.5 to about 10 mg/kg, preferably about 1 to about 5 mg/kg more preferably about 2 to about 4 mg/kg and the light delivery time is spaced in intervals of about 30 minutes to about 3 days, preferably about 12 hours to about 48 hours, and more preferably about 24 hours. The light dose administered is in the range of about 20-500 J/cm, preferably about 50 to about 300 J/cm and more preferably about 100 to about 200 J/cm. The fluence rate is in the range of about 20 to about 500 mw/cm, preferably about 50 to about 300 mw/cm and more preferably about 100 to about 200 mw/cm. There is a reciprocal relationship between photosensitizer compositions and light dose, thus, determination of suitable wavelength, light intensity, and duration of illumination is within ordinary skill in the art.

The wavelength and power of light can be adjusted according to standard methods known in the art to control the production of phototoxic species. Thus, under certain conditions (e.g., low power, low fluence rate, shorter wavelength of light or some combination thereof), a fluorescent species is primarily produced from the photosensitizer and any reactive species produced has a negligible effect. These conditions are easily adapted to bring about the production of a phototoxic species. For example, where the photosensitizer is chlorinₑ₆, the light dose administered to produce a fluorescent species and an insubstantial reactive species is less than about 10 J/cm, preferably less than about 5 J/cm and more preferably less than about 1 J/cm. Determination of suitable wavelength, light intensity, and duration of illumination for any photosensitizer is within the level of ordinary skill in the art.

D. Fluorescent Markers

Fluorescent markers of the present invention can be any known in the art, including photosensitizers, fluorescent dyes, and photoactive dyes which are optionally coupled to molecular carriers.

Fluorescent dyes of the present invention can be any known in the art, including, but not limited to 6-carboxy-4′,5′-dichloro-2′,7′-dimethoxyfluorescein succinimidyl ester; 5-(and-6)-carboxyeosin; 5-carboxyfluorescein; 6-carboxyfluorescein; 5-(and-6)-carboxyfluorescein; 5-carboxyfluorescein-bis-(5-carboxymethoxy-2-nitrobenzyl)ether, -alanine-carboxamide, or succinimidyl ester; 5-carboxyfluorescein succinimidyl ester; 6-carboxyfluorescein succinimidyl ester; 5-(and-6)-carboxyfluorescein succinimidyl ester; 5-(4,6-dichlorotriazinyl)aminofluorescein; 2′,7′-difluorofluorescein; eosin-5-isothiocyanate; erythrosin-5-isothiocyanate; 6-(fluorescein-5-carboxamido) hexanoic acid or succinimidyl ester; 6-(fluorescein-5-(and-6)carboxamido) hexanoic acid or succinimidyl ester; fluorescein-5-EX succinimidyl ester; fluorescein-5-isothiocyanate; fluorescein-6-isothiocyanate; Oregon Green® 488 carboxylic acid, or succinimidyl ester; Oregon Green® 488 isothiocyanate; Oregon Green® 488-X succinimidyl ester; Oregon Green® 500 carboxylic acid; Oregon Green® 500 carboxylic acid, succinimidyl ester or triethylammonium salt; Oregon Green® 514 carboxylic acid; Oregon Green® 514 carboxylic acid or succinimidyl ester; Rhodamine Green™ carboxylic acid, succinimidyl ester or hydrochloride; Rhodamine Green™ carboxylic acid, trifluoroacetamide or succinimidyl ester; Rhodamine Green™-X succinimidyl ester or hydrochloride; Rhodol Green™ carboxylic acid, N,O-bis-(trifluoroacetyl) or succinimidyl ester; bis-(4-carboxypiperidinyl) sulfonerhodamine or di(succinimidyl ester); 5-(and-6)carboxynaphthofluorescein, 5-(and-6)-carboxynaphthofluorescein succinimidyl ester; 5-carboxyrhodamine 6G hydrochloride; 6-carboxyrhodamine 6G hydrochloride, 5-carboxyrhodamine 6G succinimidyl ester; 6-carboxyrhodamine 6G succinimidyl ester; 5-(and-6)-carboxyrhodamine 6G succinimidyl ester; 5-carboxy-2′,4′,5′,7′-tetrabromosulfonefluorescein succinimidyl ester or bis-(diisopropylethylammonium) salt; 5-carboxytetramethylrhodamine; 6-carboxytetramethylrhodamine; 5-(and-6)-carboxytetramethylrhodamine; 5-carboxytetramethylrhodamine succinimidyl ester; 6-carboxytetramethylrhodamine succinimidyl ester; 5-(and-6)-carboxytetramethylrhodamine succinimidyl ester; 6-carboxy-X-rhodamine; 5-carboxy-X-rhodamine succinimidyl ester; 6-carboxy-X-rhodamine succinimidyl ester; 5-(and-6)-carboxy-X-rhodamine succinimidyl ester; 5-carboxy-X-rhodamine triethylammonium salt; Lissamine™ rhodamine B sulfonyl chloride; malachite green isothiocyanate; NANOGOLD® mono(sulfosuccinimidyl ester); QSY® 21 carboxylic acid or succinimidyl ester; QSY® 7 carboxylic acid or succinimidyl ester; Rhodamine Red™-X succinimidyl ester; 6-(tetramethylrhodamine-5-(and-6)-carboxamido)hexanoic acid succinimidyl ester; tetramethylrhodamine-5-isothiocyanate; tetramethylrhodamine-6-isothiocyanate; tetramethylrhodamine-5-(and-6)-isothiocyanate; Texas Reds sulfonyl; Texas Red® sulfonyl chloride; Texas Red®-X STP ester or sodium salt; Texas Red®-X succinimidyl ester; Texas Red®-X succinimidyl ester; and X-rhodamine-5-(and-6)-isothiocyanate.

Fluorescent dyes of the present invention can be, for example, BODIPY® dyes commercially available from Molecular Probes, including, but not limited to BODIPY® FL; BODIPY® TMR STP ester; BODIPY® TR-X STP ester; BODIPY® 630/650-X STP ester; BODIPY® 650/665-X STP ester; 6-dibromo-4,4-difluoro-5,7-dimethyl-4-bora-3a,4a-diaza-s-indacene-3-propionic acid succinimidyl ester; 4,4-difluoro-4-bora-3a,4a-diaza-s-indacene-3,5-dipropionic acid; 4,4-difluoro-5,7-dimethyl-4-bora-3a,4a-diaza-s-indacene-3-pentanoic acid; 4,4-difluoro-5,7-dimethyl-4-bora-3a,4a-diaza-s-indacene-3-pentanoic acid succinimidyl ester; 4,4-difluoro-5,7-dimethyl-4-bora-3a,4a-diaza-s-indacene-3-propionic acid; 4,4-difluoro-5,7-dimethyl-4-bora-3a,4a-diaza-s-indacene-3-propionic acid succinimidyl ester; 4,4-difluoro-5,7-dimethyl-4-bora-3a,4a-diaza-s-indacene-3-propionic acid sulfosuccinimidyl ester or sodium salt; 6-((4,4-difluoro-5,7-dimethyl-4-bora-3a,4a-diaza-s-indacene-3-propionyl)amino)hexanoic acid; 6-((4,4-difluoro-5,7-dimethyl-4-bora-3a,4a-diaza-s-indacene-3-propionyl)amino)hexanoic acid or succinimidyl ester; N-(4,4-difluoro-5,7-dimethyl-4-bora-3a,4a-diaza-s-indacene-3-propionyl)cysteic acid, succinimidyl ester or triethylammonium salt; 6-4,4-difluoro-1,3-dimethyl-5-(4-methoxyphenyl)-4-bora-3a,4a 4,4-difluoro-5,7-diphenyl-4-bora-3a,4a-diaza-s-indacene-3-propionic acid; 4,4-difluoro-5,7-diphenyl-4-bora-3a,4a-diaza-s-indacene-3-propionic acid succinimidyl ester; 4,4-difluoro-5-phenyl-4-bora-3a,4a-diaza-s-indacene-3-propionic acid succinimidyl ester; 6-((4,4-difluoro-5-phenyl-4-bora-3a,4a-diaza-s-indacene-3-propionyl)amino)hexanoic acid or succinimidyl ester; 4,4-difluoro-5-(4-phenyl-1,3-butadienyl)-4-bora-3a,4a-diaza-s-indacene-3-propionic acid succinimidyl ester; 4,4-difluoro-5-(2-pyrrolyl)-4-bora-3a,4a-diaza-s-indacene-3-propionic acid succinimidyl ester; 6-(((4,4-difluoro-5-(2-pyrrolyl)-4-bora-3a,4a-diaza-s-indacene-3-yl)styryloxy)acetyl)aminohexanoic acid or succinimidyl ester; 4,4-difluoro-5-styryl-4-bora-3a,4a-diaza-s-indacene-3-propionic acid; 4,4-difluoro-5-styryl-4-bora-3a,4a-diaza-s-indacene-3-propionic acid succinimidyl ester; 4,4-difluoro-1,3,5,7-tetramethyl-4-bora-3a,4a-diaza-s-indacene-8-propionic acid; 4,4-difluoro-1,3,5,7-tetramethyl-4-bora-3a,4a-diaza-s-indacene-8-propionic acid succinimidyl ester; 4,4-difluoro-5-(2-thienyl)-4-bora-3a,4a-diaza-s-indacene-3-propionic acid succinimidyl ester; 6-(((4-(4,4-difluoro-5-(2-thienyl)-4-bora-3a,4a-diaza s-indacene-3-yl)phenoxy)acetyl)amino)hexanoic acid or succinimidyl ester; and 6-(((4,4-difluoro-5-(2-thienyl)-4-bora-3a,4a-diaza-s-indacene-3-yl)styryloxy)acetyl)aminohexanoic acid or succinimidyl ester.

Fluorescent dyes the present invention can be, for example, Alexa fluor dyes commercially available from Molecular Probes, including but not limited to Alexa Fluor® 350 carboxylic acid; Alexa Fluor® 430 carboxylic acid; Alexa Fluor®1488 carboxylic acid; Alexa Fluor® 532 carboxylic acid; Alexa Fluor® 546 carboxylic acid; Alexa Fluor® 555 carboxylic acid; Alexa Fluor® 568 carboxylic acid; Alexa Fluor®1594 carboxylic acid; Alexa Fluor® 633 carboxylic acid; Alexa Fluor® 647 carboxylic acid; Alexa Fluor® 660 carboxylic acid; and Alexa Fluor® 680 carboxylic acid. Fluorescent dyes the present invention can also be, for example, cyanine dyes commercially available from Amersham-Pharmacia Biotech, including, but not limited to Cy3 NHS ester; Cy 5 NHS ester; Cy5.5 NHS ester; and Cy 7 NHS ester.

Photoactive dyes of the present invention can be any photosensitizer known in the art, which will fluoresce but not necessarily produce a reactive species in phototoxic amounts when illuminated. Depending on the wavelength and power of light administered, a photosensitizer can be activated to fluoresce and, therefore, act as a photoactive dye, but not produce a phototoxic effect unless, in some cases, the wavelength and power of light is suitably adapted to induce a phototoxic effect.

E. Radiolabeled Markers

Radiolabeled markers of the present invention can comprise any known in the art, including, but not limited to radionuclide or a paramagnetic contrast agents, preferably beta-emitting agents, which are optionally coupled to molecular carriers.

Examples of appropriate radionuclides for use in radiolabeling include, but are not limited to ¹³¹I, ¹²⁵I, ¹²³I, ^{99m}Tc, ¹⁸F, ⁶⁸Ga, ⁶⁷Ga, ⁷²As, ⁸⁹Zr, ⁶²Cu, ¹¹¹Cu, ²⁰³In, ¹⁹⁸Pb, ¹⁹⁸Hg, ⁹⁷Ru, ¹¹C, ¹⁸⁸Re, and ²⁰¹Tl. Suitable paramagnetic contrast agents include, but are not limited to gadolinium, cobalt, nickel, manganese and iron.

In a specific embodiment, the diagnostic agent is a β-emitter, for example, ¹⁸F-Fluorodeoxyglucose (“FDG”). Other β-emitters include but are not limited to ¹³¹I, ¹⁸⁶Re, which is electron emitting, or ¹⁸⁸Re, which is positron emitting. β-detecting devices distinguish β rays from γ rays by a ratio of about 100:1 (i.e., 100:1 β to γ), more preferably by a ratio of 1000:1 (i.e, 1000:1 β to γ).

Detection of radiolabeled compositions can comprise imaging or standard means known in the art. For example, radionuclides or paramagnetic contrast agents can be detected by gamma detecting devices. One of ordinary skill in the art will appreciate that the methods of detecting positrons or γ-photons, as well as radionuclides, will require different detection techniques.

Imaging methods such as magnetic resonance imaging (MRI), and computer tomography (CT), are widely used because of their ability to non-invasively image body organs and tissues with minor deleterious effects. In these techniques, an organ or tissue is irradiated with electromagnetic waves. The waves reflected or scattered by the organ or tissue are recorded and processed into a digital image.

Generally, MRI is a well-known imaging technique. A conventional MRI device establishes a homogenous magnetic field, for example, along an axis of a person's body that is to undergo MRI. This homogeneous magnetic field conditions the interior of the person's body for imaging by aligning the nuclear spins of nuclei (in atoms and molecules forming the body tissue) along the axis of the magnetic field. For discussions on in vivo nuclear magnetic resonance imaging, see, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415.

Positron Emission Tomography (PET) and Single Photon Emission Computed Tomography (SPECT) are imaging techniques in which a radionuclide is synthetically introduced into a molecule of potential biological significance, such as a tracer. The subsequent uptake of the radiotracer is measured over time and used to obtain information about the physiological process of interest. While PET and SPECT rely on similar principles to produce their images, differences in instrumentation, radiochemistry, and experimental applications are are accounted for by differences in their respective physics of photon emission.

Unstable nuclides that possess an excess number of protons may take one of two approaches in an effort to reduce their net nuclear positivity. In one radioactive decay scheme, a proton is converted to a neutron and a particle called a positron is emitted (Hoffman, E. J., and Phelps, M. E. New York: Raven Press; 1986: 237-286; Sorenson, J. A., and Phelps, M. E. Philadelphia: W.B. Saunders; 1987). Of identical mass but opposite charge, positrons are the antimatter equivalent of electrons. When ejected from the nucleus, a positron collides with an electron, resulting in the annihilation of both particles and the release of energy. Two γ photons are produced, each of equivalent energy and opposite trajectory (generally 180° apart).

The unique spatial signature of back-to-back photon paths is exploited by PET scanners in locating the source of an annihilation event, a method known as coincidence detection (Hoffman, E. J., and Phelps, M. E. New York: Raven Press; 1986: 237-286; Links, J. M. New York: Raven Press; 1990: 37-50). PET (and SPECT) scanners employ scintillation detectors made of dense crystalline materials (e.g., bismuth germanium oxide, sodium iodide, or cesium fluoride), that capture the high-energy photons and convert them to visible light. This brief flash of light is converted into an electrical pulse by an adjacent photomultiplier tube (PMT). The crystal and PMT together make up a radiation detector. A PET camera is constructed such that opposing detectors are electronically connected. Thus, when separate scintillation events in paired detectors coincide, an annihilation event is presumed to have occurred at some point along an imaginary line between the two. This information is used to reconstruct images using the principles of computed tomography.

Isotopes that decay by electron capture and/or γ emissions can be directly detected by SPECT. Certain proton-rich radionuclides, such as ¹²³I and ^{99m}Tc, may instead capture an orbiting electron, once again transforming a proton to a neutron (Sorenson J A, and Phelps M E. Philadelphia: W.B. Saunders; 1987). The resulting daughter nucleus often remains residually excited. This meta-stable arrangement subsequently dissipates, thereby achieving a ground state and producing a single γ photon in the process. Because γ photons are emitted directly from the site of decay, no comparable theoretical limit on spatial resolution exists for SPECT. However, instead of coincidence detection, SPECT utilizes a technique known as collimation (Jaszczak R J. Boca Raton: CRC Press; (1991): 93-118). A collimator may be thought of as a lead block containing many tiny holes that is interposed between the subject and the radiation detector. Given knowledge of the orientation of a collimator's holes, the original path of a detected photon is linearly extrapolated and the image is reconstructed by computer-assisted tomography.

Radiolabeled markers of the invention can be used in accordance with the methods of the invention by those of skill in the art to image inflamed tissue in a subject. Images are generated by virtue of differences in the spatial distribution of the compositions that accumulate in the various tissues and organs of the subject. The spatial distribution of the imaging agent accumulated can be measured using devices of the present invention. Background signal is evident when a less intense signal is detected, indicating the presence of tissue in which a lower concentration of a radiolabeled composition accumulates relative to the concentration of the same, which accumulates in the inflamed tissue.

Accordingly, inflamed tissue can be detected as a more intense signal, indicating a region of enhanced concentration of the radiolabeled composition at the site relative to the concentration of the same which accumulates elsewhere. The extent of accumulation of the radiolabeled composition can be quantified using known methods for quantifying radioactive emissions. A particularly useful imaging approach to employs more than one imaging agent to perform simultaneous studies.

F. Molecular Carriers

Enhanced selectivity for inflammed tissues can be achieved by using covalent conjugates or non-covalent complexes between molecular carriers having targeting specificity for inflammatory cells or other inflammatory components located in close proximity to inflammatory cells. Accordingly, diagnostic and therapeutic compositions of the present invention can comprise diagnostic or therapeutic agents “coupled” to molecular carriers. Use of molecular carriers allows, for example, a photosensitizer to be selected according to optical and photophysical properties, without relying on the molecular structure of the photosensitizer to provide a tissue-selective effect (Hasan, T. (1992) In: B. Henderson and T. Dougherty (eds.), Photodynamic Therapy Basic Principles and Clinical Applications. pp. 187-200: Marcel Dekker).

Generally, molecular targeting is based on two facets of molecular structure. First features of the molecular carriers such as size, charge, hydrophobicity and biodegradability can be manipulated to increase accumulation or retention in the inflamed tissue, and, second, the molecular carrier can be designed to recognize antigens, receptors or other cell type specific structures present on inflammatory cells or other inflammatory components. In specific embodiments, the molecular carrier comprises serum proteins including receptor ligands (Hamblin et al. (1994) J. Photochem. Photobiol. 26:147-157; Hamblin and Newman (1994) J. Photochem. Photobiol. 26:45-56), microspheres (Bachor et al. (1991) Proc. Natl. Acad. Sci. U.S.A. 88:1580-1584), liposomes (Polo et al. (1996) Cancer Lett. 109:57-61), polymers (Hamblin et al. (1999) Br. J. Cancer 81:261-268), monoclonal antibodies (Hamblin et al. (2000) Br. J. Cancer 83:1544-1551), growth factors (Gijsens and De Witte (1998) Int. J. Oncol. 13:1171-1177), peptides (Krinick, (1994) J. Biomater. Sci. Polym. Ed. 5: 303-324), hormones (Akhlynina et al. (1995) Cancer Res. 55:1014-1019) and lipoproteins (Schmidt-Erfurth et al. (1997) Br. J. Cancer 75:54-61).

In a specific embodiment, therapeutic and diagnostic agents of the present invention are coupled to molecular carriers comprising ligands that bind to “scavenger receptors.” Scavenger receptors are membrane proteins expressed on the surface of macrophages, monocytes, endothelial cells and smooth muscle cells that recognize a wide range of ligands, both naturally occurring and synthetic (Freeman et al. (1997) Curr. Opin. Hematol. 4:41-47). Presently, there are six members of the scavenger receptor family belonging to three classes (e.g., class A, B or C). After initial binding to the scavenger receptor, the ligands are rapidly internalized and are routed to lysosomes for degradation by proteases and other lysosomal enzymes. The wide and diverse range of structures recognized by these receptors has led to them being termed “molecular flypaper” (Krieger et al. (1992) Trends Biochem. Sci 17:141-146, 1992). The ligands are all molecules with a pronounced anionic charge that have some common conformational features (Haberland and Fogelman (1985) Proc. Natl. Acad. Sci. U.S.A., 82:2693-2697; Takata (1989) Biochem. Biophys. Acta. 984:273-280). Specific targeting of compositions to J774 and other macrophage-like cells in vitro has been achieved with conjugates of maleylated albumin, daunorubicin and doxorubiciri (Mukhopadhyay et al (1992) Biochem J. 284:237-241; Basu et al. (1994) FEBS Lett. 342:249-254; Hamblin et al. (2000) Photochem Photobiol. 4:533-540).

Numerous scavenger receptor ligands known in the art (either with or without polyethyl glycolization) can be used to localize therapeutic and diagnostic compositions of the present invention to inflamed tissues, including, but not limited to glucose analogs (e.g. fluorodeoxyglucose), chemotactic peptide receptor agonist anologs, maleylated albumin, oxidized low density lipoprotein, acetylated low density lipoprotein, oxidized high density lipoprotein, lipopolysaccharide, malondialdehyde treated proteins, lipotechoic acid, formaldehyde treated albumin, glycated albumin, polyinosinic acid, glycated lipoproteins, dextran sulfate, anionic phospholipids (phosphatidylserine), fucoidin, carrageenan, polyvinyl sulfate, monoclonal antibodies that recognize CD11b or c, CD13, CD14, CD16a, CD32, or CD68, polyvinyl sulfate, crocidolite asbestos.

In a specific embodiment, therapeutic and diagnostic agents of the present invention are coupled to molecular carriers that target macrophages and/or monocytes of inflamed tissues. These molecular carriers can be targeted to, for example, tenascin C, tissue factor, tissue inhibitor of MMP 1 and 2, oxidized LDL receptor (also known in the art as CD36), heme oxygenase-1, human cartilage gp-39, IL-6, IL-6 receptor, IL-10, IL-10 receptor, lectin-like oxidized LDL-receptor (“LOX-1”), bacterial chemotactic peptide receptor agonists, preferably Formyl-Methionine-Leucine-Phenylalanine (“F-MLP”), macrophage chemoattractant protein-1 receptor (“CCR-9”) and monocyte inflammatory protein-1 and receptors thereof (including “CCR-5”). Such molecular carriers can be, for example, antibodies against these biomolecules, ligands binding the same or analogs thereof.

In a specific embodiment, therapeutic and diagnostic agents of the present invention are coupled to molecular carriers that target T cells of inflammed tissues. These molecular carriers can be targeted to, for example, IL-10, IL-10 receptor, monocyte inflammatory protein-1 and receptors thereof and transferrin. Such molecular carriers can be, for example, antibodies against these biomolecules, ligands binding the same or analogs thereof, including, but not limited to monoclonal antibodies that recognize CD1, CD2, CD3, CD4, CD5, CD6, CD7, CD8, CD25, CD28, CD44, CD71 or transferrin.

In a specific embodiment, therapeutic and diagnostic agents of the present invention are coupled to molecular carriers that target proteases that degrade extracellular matrix (e.g., metalloproteinases), including but not limited to monoclonal antibodies against the protease and proteinase substrates.

In a specific embodiment, therapeutic and diagnostic agents of the present invention are coupled to molecular carriers that target the endothelial cells of inflammed tissues. These molecular carriers can be targeted to, for example, endothelial adhesion molecules including, but not limited to, ICAM (also known in the art as CD54) and VCAM (also known in the art as CD106), angiotensin II, angiotensin converting enzyme (also known in the art as CD143), endothelial derived lipase, tissue factor, heme oxygenase-1, LOX-1, low density lipoprotein (“LDL”), high density lipoprotein, (“HDL”), P-selectin, L-selectin and E-selectin. Such molecular carriers can be, for example, antibodies against these biomolecules, ligands binding the same or analogs thereof.

In a specific embodiment, therapeutic and diagnostic agents of the present invention are coupled to molecular carriers that target neutrophils of inflammed tissues. These molecular carriers can be targeted to, for example, myeloperoxidase. Such molecular carriers can be, for example, antibodies against these biomolecules, ligands binding the same or analogs thereof.

In a specific embodiment, therapeutic and diagnostic agents of the present invention are coupled to molecular carriers that target B cells of inflammed tissues. These molecular carriers can be targeted to, for example, IL-6, IL-6 receptor, IL-10 and IL-10 receptor. Such molecular carriers can be, for example, antibodies against these biomolecules, ligands binding the same or analogs thereof.

In a specific embodiment, therapeutic and diagnostic agents of the present invention are coupled to molecular carriers that either directly or indirectly associate with the target. For example, indirect targeting can be achieved by first localizing a biotinylated molecular carrier to a target, followed by administration of a streptavidin-linked composition comprising, for example, a photoactive dye, fluorescent dye, photosensitizer or radioactive agent.

Thus, localizing a therapeutic or diagnostic composition to activated macrophages or proteases that degrade extracellular matrix via a molecular carrier, for example, confers a selective advantage on an inflammed tissue, such that uptake of the composition is far greater than in non-inflammed tissue.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

Compositions of the present invention that are useful for detection of inflammed tissues can include radiolabeled molecular carriers. A number of radiolabeled molecular carriers have been tested for their ability to bind to and permit scintigraphic detection of atherothrombotic materials. These include labeled antibodies to oxidized LDL, fibrinogen, autologous platelets, fibrin fragment E1, plasminogen activators, and ^{99m}Tc-conjugated antibodies against modified LDL (Tsimikas et al. (1999) J. Nucl. Cardiol. 6: 41-53).

Such radiolabels can be associated with the molecular carrier by ionic association or covalent bonding directly to an atom of the carrier. The radiolabel can be non-covalently or covalently associated with the carrier through a chelating structure. A “chelating structure” refers to any molecule or complex of molecules that bind to both the label and targeting moiety. Many such chelating structures are known in the art. Chelating structures include, but are not limited to —N₂S₂, —NS₃, —N₄, dota derivatives [1,4,7,10-tetrakis(carboxymethyl)-1,4,7,10-tetrazacyclododecane], an isonitrile, a hydrazine, a HYNIC (hydrazinonicotinic acid), 2-methylthiolnicotinic acid, phosphorus, or a carboxylate containing group; or through an auxiliary molecule such as mannitol, gluconate, glucoheptonate, tartrate, and the like. In some cases, chelation can be achieved without including a separate chelating structure, because the radionuclide chelates directly to atom(s) in the molecular carrier, for example to oxygen atoms in various moieties.

The chelating structure, auxiliary molecule, or radionuclide may be placed in spatial proximity to any position of the molecular carrier that does not interfere with the interaction of the targeting molecule with its target site in cardiovascular tissue. Accordingly, the chelating structure, auxiliary molecule, or radionuclide may be covalently or non-covalently associated with any moiety of the molecular carrier (except the receptor-binding moiety where the molecular carrier is a receptor and the epitope-binding region where the molecular carrier is an antibody).

Radionuclides can be placed in spatial proximity to the molecular carrier using known procedures that effect or optimize chelation, association, or attachment of the specific radionuclide to ligands. For example, when ¹²³I is the radionuclide, the imaging agent may be labeled in accordance with the known radioiodination procedures such as direct radioiodination with chloramine T, radioiodination exchange for a halogen or an organometallic group, and the like. When the radionuclide is ^{99m}Tc, the imaging agent may be labeled using any method suitable for attaching ^{99m}Tc to a ligand molecule. Preferably, when the radionuclide is ^{99m}Tc, an auxiliary molecule such as mannitol, gluconate, glucoheptonate, or tartrate is included in the labeling reaction mixture, with or without a chelating structure. More preferably, ^{99m}Tc is placed in spatial proximity to the carrier by reducing ^{99m}TcO₄, with tin in the presence of mannitol and the targeting molecule. Other reducing agents, including tin tartrate or non-tin reductants such as sodium dithionite, may also be used to make radiolabeled compositions of the present invention.

In general, labeling methodologies vary with the choice of radionuclide and the carrier to be labeled. Labeling methods are described, for example, in Peters et al. (1986) Lancet 2:946-949; Srivastava et al. (1984) Semin. Nucl. Med. 14:68-82; Eckelman and Richards (1972) J. Nucl. Med. 13:180; McAfee et al. (1976) J. Nucl. Med. 17:480-487; Welch et al., (1977) J. Nucl. Med. 18:558-562; Thakur et al. (1984) Semin. Nucl. Med. 14:107; Danpure et al. (1981) Br. J. Radiol. 54:597-601; Danpure et al. (1982) Br. J. Radiol. 55:247-249; Peters et al. (1983) J. Nucl. Med. 24:39-44; Gunter et al. (1983) Radiology 149:563-566 and Thakur et al. (1985) J. Nucl. Med. 26:518-523.

After the labeling reaction is complete, the reaction mixture may optionally be purified using one or more chromatography steps such as Sep Pak or high performance liquid chromatography (HPLC). Any suitable HPLC system may be used if a purification step is performed, and the yield of cardiovascular imaging agent obtained from the HPLC step may be optimized by varying the parameters of the HPLC system, as is known in the art. Any HPLC parameter may be varied to optimize the yield of the cardiovascular imaging agent of the invention. For example, the pH may be varied, e.g., raised to decrease the elution time of the peak corresponding to the radiolabeled carrier.

Photosensitizers can also be coupled to a molecular carrier, such as a scavenger receptor ligand, either directly or indirectly via a “backbone” or “bridge” moiety, such as a polyamino acid, whereby the backbone is coupled both to the photosensitizer and the molecular carrier.

Inclusion of a backbone in a composition with a photosensitizer and a molecular carrier can provide a number of advantages, including the provision of greater stoichiometric ranges of photosensitizer and molecular carriers coupled per backbone. If the backbone possesses intrinsic affinity for a target organism, coupling to the backbone can enhance the affinity of the composition. Coupling two or more different molecular carriers to a single photosensitizerbackbone composition can expand the specific range of cells that can be targeted with one composition.

Peptides useful in the methods of the invention for design and characterization of backbone moieties include poly-amino acids which can be homo- and hetero-polymers of L-, D-racemic DL- or mixed L- and D-amino acid composition, and which can be of defined or random mixed composition and sequence. These peptides can be modeled after particular natural peptides, and optimized by the technique of phage display and selection for enhanced binding to a chosen target, so that the selected peptide of highest affinity is characterized and then produced synthetically. Further modifications of functional groups can be introduced for purposes, for example, of increased solubility, decreased aggregation, and altered extent of hydrophobicity. Examples of nonpeptide backbones include nucleic acids and derivatives of nucleic acids such as DNA, RNA and peptide nucleic acids; polysaccharides and derivatives such as starch, pectin, chitins, celluloses and hemi-methylated celluloses; lipids such as triglyceride derivatives and cerebrosides; synthetic polymers such as polyethylene glycols (PEGS) and PEG star polymers; dextran derivatives, polyvinyl alcohols, N-(2-hydroxypropyl)methacrylamide copolymers, poly (DL-glycolic acid-lactic acid); and compositions containing elements of any of these classes of compounds.

Modifying the charge of a component of the composition can refine the affinity of a photosensitizer composition. Conjugates such as poly-L-lysine chlorinₑ₆ can be made in varying sizes and charges (cationic, neutral, and anionic), for example, free NH2 groups of the polylysine are capped with acetyl, succinyl, or other R groups to alter the charge of the final composition. Net charge of a composition of interest can be determined by isoelectric focusing (IEF). This technique uses applied voltage to generate a pH gradient in a non-sieving acrylamide or agarose gel by the use of a system of ampholytes (synthetic buffering components). When charged polypeptides are applied to the gel they will migrate either to higher pH or to lower pH regions of the gel according to the position at which they become uncharged and hence unable to move further. This position can be determined by reference to the positions of a series of known IEF marker proteins.

In a specific embodiment, diagnostic and therapeutic compositions of the present invention can comprise diagnostic and therapeutic agents coupled molecular carriers that are antibodies. For example, photosensitizers coupled to antibodies are known in the art as “photoimmunoconjugates.” The antibody component of the composition can bind with specificity to an epitope present on the surface of an inflammatory cell associated with an inflamed tissue. As used herein, the term “binding with specificity” means that the antibody only poorly recognizes cells that do not express the epitope.

The term “antibody” as used herein includes intact molecules as well as fragments thereof, such as Fab and Fab′, which are capable of binding the epitopic determinant. Fab fragments retain an entire light chain, as well as one-half of a heavy chain, with both chains covalently linked by the carboxy terminal disulfide bond. Fab fragments are monovalent with respect to the antigen-binding site. Antibodies that can be used in the methods of the present invention can comprise whole native antibodies, bispecific antibodies; chimeric antibodies; Fab, Fab′, single chain variable region fragments (scFv) and fusion polypeptides. Preferably, the antibodies are monoclonal.

Antibodies can be prepared in several ways. Methods of producing and isolating whole native antibodies, bispecific antibodies; chimeric antibodies; Fab, Fab′, single chain V region fragments (scFv) and fusion polypeptides are known in the art. See, for example, Harlow and Lane (1988) Antibodies: A Laboratory Manual, Cold Spring Harbor Laboratory, New York (Harlow and Lane, 1988).

Antibodies are most conveniently obtained from hybridoma cells engineered to express an antibody. Methods of making hybridomas are well known in the art. The hybridoma cells can be cultured in a suitable medium, and spent medium can be used as an antibody source. Polynucleotides encoding the antibody can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition, e.g., Pristane.

Another method of obtaining antibodies is to immunize suitable host animals with an antigen and to follow standard procedures for polyclonal or monoclonal production. Monoclonal antibodies (Mabs) thus produced can be “humanized” by methods known in the art. Examples of humanized antibodies are provided, for instance, in U.S. Pat. Nos. 5,530,101 and 5,585,089.

“Humanized” antibodies are antibodies in which at least part of the sequence has been altered from its initial form to render it more like human immunoglobulins. In one version, the heavy chain and light chain C regions are replaced with human sequence. In another version, the CDR regions comprise amino acid sequences for recognition of antigen of interest, while the variable framework regions have also been converted to human sequences. See, for example, EP 0329400. In a third version, variable regions are humanized by designing consensus sequences of human and mouse variable regions, and converting residues outside the CDRs that are different between the consensus sequences. The invention encompasses humanized Mabs.

The invention also encompasses hybrid antibodies, in which one pair of heavy and light chains is obtained from a first antibody, while the other pair of heavy and light chains is obtained from a different second antibody. Such hybrids may also be formed using humanized heavy and light chains.

Construction of phage display libraries for expression of antibodies, particularly the Fab or scFv portion of antibodies, is well known in the art (Heitner et al. (2001) J Immunol Methods 248:17-30). The phage display antibody libraries that express antibodies can be prepared according to the methods described in U.S. Pat. No. 5,223,409 incorporated herein by reference. Procedures of the general methodology can be adapted using the present disclosure to produce antibodies of the present invention. The method for producing a human monoclonal antibody generally involves (1) preparing separate heavy and light chain-encoding gene libraries in cloning vectors using human immunoglobulin genes as a source for the libraries, (2) combining the heavy and light chain encoding gene libraries into a single dicistronic expression vector capable of expressing and assembling a heterodimeric antibody molecule, (3) expressing the assembled heterodimeric antibody molecule on the surface of a filamentous phage particle, (4) isolating the surface-expressed phage particle using immunoaffinity techniques such as panning of phage particles against a preselected antigen, thereby isolating one or more species of phagemid containing particular heavy and light chain-encoding genes and antibody molecules that immunoreact with the preselected antigen.

Linking light and heavy chain variable regions by using a short linking peptide makes single chain variable region fragments. Any peptide having sufficient flexibility and length can be used as a linker in a scFv. Usually the linker is selected to have little to no immunogenicity. An example of a linking peptide is (GGGGS)₃, which bridges approximately 3.5 nm between the carboxy terminus of one variable region and the amino terminus of another variable region. Other linker sequences can also be used. All or any portion of the heavy or light chain can be used in any combination. Typically, the entire variable regions are included in the scFv. For instance, the light chain variable region can be linked to the heavy chain variable region. Alternatively, a portion of the light chain variable region can be linked to the heavy chain variable region or a portion thereof. Also contemplated are compositions comprising a biphasic scFv in which one component is a polypeptide that recognizes an antigen and another component is a different polypeptide that recognizes a different antigen, such as a T cell epitope.

ScFvs can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect or mammalian cells, or prokaryotic, such as Escherichia coli, and the protein expressed by the polynucleotide can be isolated using standard protein purification techniques.

A particularly useful system for the production of scFvs is plasmid pET-22b(+) (Novagen, Madison, Wis.) in E. coli. pET-22b(+) contains a nickel ion binding domain consisting of 6 sequential histidine residues, which allows the expressed protein to be purified on a suitable affinity resin. Another example of a suitable vector is pcDNA3 (Invitrogen, San Diego, Calif.), described above.

Expression conditions should ensure that the scFv assumes functional and, preferably, optimal tertiary structure. Depending on the plasmid used (especially the activity of the promoter) and the host cell, it may be necessary or useful to modulate the rate of production. For instance, use of a weaker promoter, or expression at lower temperatures may be necessary or useful to optimize production of properly folded scFv in prokaryotic systems; or, it may be preferable to express scFv in eukaryotic cells. Antibody purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Therapeutic and diagnostic agents can be linked to antibodies according to any method known in the art. For example, the antibody can be directly linked to the agent through a polymer or a polypeptide linkage. Polymers of interest include, but are not limited to polyamines, polyethers, polyamine alcohols, derivitized to components by means of ketones, acids, aldehydes, isocyanates or a variety of other groups. Polypeptide linkages can comprise, for example poly-L-lysine linkages (Del Governatore et al. (2000) Br. J. Cancer 82:56-64; Hamblin et al. (2000) Br. J. Cancer 83:1544-41; Molpus et al. (2000) Gynecol Oncol 76:397-404). In a specific embodiment, the antibody can be linked to a photosensitizer and at least one solubilizing agent each of which are independently bound to the antibody through a direct covalent linkage. The direct covalent linkage can be, for example, an amide linkage to a lysine residue of the antibody, as described in U.S. application number 20020197262 (Ser. No. 10/137,029; published May 1, 2002), the contents of which are herein incorporated by reference.

### III. Administration

An “effective amount” of a therapeutic composition, diagnostic composition, or immune modulator is an amount sufficient to effect a beneficial or desired clinical result. An effective amount can be administered in one or more doses. In terms of treatment, an effective amount is an amount that is sufficient to palliate, ameliorate, stabilize, reverse or slow the progression of inflammation characterized by the presence of immunoregulatory cells or otherwise reduce the pathological consequences of the inflammatory response. The effective amount is generally determined by the physician on a case-by-case basis and is within the skill of one in the art.

As a rule, the dosage for in vivo therapeutics or diagnostics will vary. Several factors are typically taken into account when determining an appropriate dosage. These factors include age, sex and weight of the patient, the condition being treated, and the severity of the condition.

Suitable dosages and formulations of immune modulators can be empirically determined by the administering physician. Standard texts, such as Remington: The Science and Practice of Pharmacy, 17th edition, Mack Publishing Company, and the Physician's Desk Reference, each of which are incorporated herein by reference, can be consulted to prepare suitable compositions and doses for administration. A determination of the appropriate dosage is within the skill of one in the art given the parameters for use described herein.

Administration can be in any order. For example, the diagnostic agent can be administered before, after or during administration of the immune modulator. The therapeutic agent can also be administered before, after or during administration of the immune modulator.

In accordance with the invention, “an effective amount of the radiolabeled composition” of the invention is defined as an amount sufficient to yield an acceptable signal using equipment that is available for clinical use. An effective amount of the radiolabeled composition of the invention can be administered in more than one dose. Effective amounts of the radiolabeled composition of the invention will vary according to factors such as the degree of susceptibility of the individual, the age, sex, and weight of the individual, idiosyncratic responses of the individual, and the dosimetry. Effective amounts of the imaging agent of the invention will also vary according to instrument and film-related factors. Optimization of such factors is well within the level of skill in the art. In general, the effective amount will be in the range of from about 0.1 to about 10 mg by injection or from about 5 to about 100 mg orally.

Radiolabeled markers of the present invention, optionally coupled to molecular carriers or molecular carriers and photosensitizers, can comprise, for example, from about 1 to about 30 mCi of the radionuclide in combination with a pharmaceutically acceptable carrier. Such compositions may be provided in solution or in lyophilized form. Suitable sterile and physiologically acceptable reconstitution media include water, saline, buffered saline, and the like. Radionuclides can be combined with the unlabeled molecular carrier/chelating agent and a reducing agent for a sufficient period of time and at a temperature sufficient to chelate the radionuclide to the molecular carrier prior to injection into the patient.

The radiolabeled compositions, optionally comprising molecular carriers or molecular carriers and photosensitizers, can be administered to a subject in accordance with any means that facilitates accumulation of the agent in a subject's cardiovascular system. For example, the radiolabeled composition of the invention is administered by arterial or venous injection, and has been formulated as a sterile, pyrogen-free, parenterally acceptable aqueous solution. The preparation of such parenterally acceptable solutions, having due regard to pH, isotonicity, stability, and the like, is within the skill in the art. A typical formulation for intravenous injection contains an isotonic vehicle such as Sodium Chloride Injection, Ringer's Injection, Dextrose Injection, Dextrose and Sodium Chloride Injection, Lactated Ringer's Injection, or other vehicle as known in the art.

The amount of radiolabeled composition used for diagnostic purposes and the duration of the study will depend upon the nature and severity of the condition being treated, on the nature of therapeutic treatments which the patient has undergone, and on the idiosyncratic responses of the patient. Ultimately, the attending physician will decide the amount of radiolabeled composition to administer to each individual patient and the duration of the imaging study.

The dosage of fluorescent markers or photosensitizers can range from about 0.1 to about 10 mg/kg. Methods for administering fluorescent compositions are known in the art, and are described, for example, in U.S. Pat. Nos. 5,952,329, 5,807,881, 5,798,349, 5,776,966, 5,789,433, 5,736,563, 5,484,803 and by (Sperduto et al. (1991) Int. J. Radiat. Oncol. Biol. Phys. 21:441-6; Walther et al. (1997) Urology 50:199-206). Such dosages may vary, for example, depending on whether multiple administrations are given, tissue type and route of administration, the condition of the individual, the desired objective and other factors known to those of skill in the art. Where the fluorescent compositions comprises a photosensitizer conjugated to an antibody, or a “photoimmunoconjugate,” dosages can vary from about 0.01 mg/m² to about 500 mg/m², preferably about 0.1 mg/m² to about 200 mg/m², more preferably about 0.1 mg/m² to about 10 mg/m². Ascertaining dosage ranges is well within the skill of one in the art. For instance, the concentration of scFv typically need not be as high as that of native antibodies in order to be therapeutically effective. Administrations can be conducted infrequently, or on a regular weekly basis until a desired, measurable parameter is detected, such as diminution of disease symptoms. Administration can then be diminished, such as to a biweekly or monthly basis, as appropriate.

Following administration of the diagnostic composition, it can be necessary to wait for the composition to reach an effective tissue concentration at the site of the inflammation before detection. Duration of the waiting step varies, depending on factors such as route of administration, location, and speed of movement in the body. In addition, where the compositions are coupled to molecular carriers, the rate of uptake can vary, depending on the level of receptor expression on the surface of the cells. For example, where there is a high level of receptor expression, the rate of binding and uptake is increased. Determining a useful range of waiting step duration is within the level of ordinary skill in the art and may be optimized by utilizing fluorescence optical imaging techniques.

Compositions of the present invention are administered by a mode appropriate for the form of composition. Available routes of administration include subcutaneous, intramuscular, intraperitoneal, intradermal, oral, intranasal, intrapulmonary (i.e., by aerosol), intravenously, intramuscularly, subcutaneously, intracavity, intrathecally or transdermally, alone or in combination with other pharmaceutical agents. Therapeutic compositions of photosensitizers are often administered by injection or by gradual perfusion.

Compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, compositions are preferably supplied in unit dosage form suitable for administration of a precise amount. Also contemplated by this invention are slow-release or sustained release forms, whereby a relatively consistent level of the active compound are provided over an extended period.

Another method of administration is intravascular, for instance by direct injection into the blood vessels of the inflamed tissue or surrounding area.

Further, it may be desirable to administer the compositions locally to the area in need of treatment; this can be achieved, for example, by local infusion during surgery, by injection, by means of a catheter, or by means of an implant, said implant being of a porous, non-porous, or gelatinous material, including membranes, such as silastic membranes, or fibers. A suitable such membrane is Gliadel® provided by Guilford Pharmaceuticals Inc.

The present invention is additionally described by way of the following illustrative, non-limiting Examples that provide a better understanding of the present invention and of its many advantages.

### IV. Inflammed Tissues and Associated Disorders

Within the scope of the invention, an adverse immune response can produce an inflamed tissue. The adverse immune response can be attributed to various diseases and conditions that affect the tissues of one or more organs or organ systems including, but not limited to, the peripheral nervous system, hematological system, bone marrow, the central nervous system, skin, appendix, gastrointestinal tract (including but not limited to esophagus, duodenum, and colon), respiratory/pulmonary system (including but not limited to lung, nose, pharynx, larynx), eye, genito-reproductive system, gums, liver/biliary ductal system, renal system (including but not limited to kidneys, urinary tract, bladder), connective tissue (including but not limited to joints, cartilage), cardiovascular system, muscle, heart, spleen, breast, lymphatic system, ear, endocrine/exocrine system (including but not limited to lacrimal glands, salivary glands, thyroid gland, pancreas), and bone/skeletal system.

An adverse immune response can be triggered as a result of an inflammatory disease. Inflammatory diseases that affect the peripheral nervous system include, but are not limited to, radiculitis. Inflammatory diseases of the central nervous system include acute hemorrhagic leukoencephalitis, cholesterol granuloma, meningoencephalitis, optic neuritis, and Parsonage-Aldren-Turner syndrome, but are not limited to these diseases. Inflammatory disease s of the skin can include, but are not limited to, acute infantile hemorrhagic edema, contact dermatitis, Favre-Racouchot syndrome, folliculitis, panniculitis, Riehl's melanosis, Stevens-JohrLson syndrome, and trichostasis spinulosa. Inflammatory diseases of the appendix include appendicitis, Atrophic gastritis, Barrett's esophagus, Celiac disease, colitis, colonic diverticulitis, Curling's ulcers, Cushing's ulcers, esophagitis, phlegmonous gastritis, proctitis, toxic megacolon, and typhlitis are some inflammatory diseases that affect the gastrointestinal tract. Inflammatory diseases of the respiratory/pulmonary system include, but are not limited to atrophic rhinitis, bronchiolitis obliterans organizing pneumonitis, pleural empyema, endogenous lipoid pneumonia, laryngeal granuloma, lymphocytic interstitial pneumonia, pharyngitis, pleuritis, sinusistis, and sterile pneumonitis. Inflammatory diseases of the eye can be blepharitis, dacryocystitis, endophthalmitis, Fuch's heterochromic cyclitis, giant papillary conjunctivitis, optic neuritis, phlyctenular keratoconjunctivitis, scleritis, but are not limited to these examples.

The allergic reaction has been extensively studied and the basic immune mechanisms involved are well known.

Diseases characterized by inflammation that affect the genito-reproductive system include, but are not limited to Bowenoid papulosis, cervicitis, cystitis, epidydymo-orchitis, peritonitis, and prostatitis. Inflammatory diseases that affect the gums include cancrum oris, giant cell granuloma, gingivitis, pericoronitis, periodontitis, and pulpitis, but are not limited to these examples. Diseases states that are characterized by inflammation and that affect the liver/biliary ductal system include, but are not limited to, cholangitis and perihepatitis. Inflammatory diseases of the renal system can include chronic interstitial nephritis, Hunner's ulcer, post-streptococcal glomerulonephritis, and xanthogranulomatous pyelonephritis. Disease states that affect connective tissue include, but are not limited to, De Quervain's tenosynovitis, pyrophosphate arthropathy, reactive arthropathy, sacroilitis, synovitis, tenosynovitis, Tietze's costochondritis, and urate crystal arthropathy.

Disease states characterized by inflammation of the cardiovascular system include endocarditis, pericarditis, thrombophlebitis, and vasculitis, but are not limited to these examples. Inflammatory disease states that affect muscle include but are not limited to, myositis and Parsonage-Aldren-Turner syndrome. Mastitis and Mondor's disease of the breast are some inflammatory conditions that affect the breast. Diseases of the lymphatic system that are characterized by inflammation include mesenteric adenitis and pseudolymphoma, but are not limited to these examples. Inflammatory diseases of the ear can include diseases such as myringitis bullosa. Inflammatory diseases of the endocrine/exocrine system can include necrotizing sialometaplasia, pancreatitis, parotitis, and thyroiditis, while diseases of the bone/skeletal system characterized by inflammation include osteitis, osteitis fibrosa cystica, osteitis pubis, and periostitis, but are not limited to these examples. It is evident that many inflammatory diseases can be systemic and affect more than one organ system. Some systemic inflammatory diseases can include gangrene, Jarisch-Herxheimer reaction, and Reiter's syndrome.

Methods and compositions of the invention are also suitable for the detection and therapy of adverse immune responses comprising autoimmune responses. Autoimmune disease is a class of diseases in which a subject's own antibodies react with host tissue or in which immune effector T cells are autoreactive to endogenous self-peptides and cause destruction of tissue. Autoimmune diseases include, but are not limited to, acquired factor VIII deficiency, acquired generalized lipodystrophy, alopecia areata, ankylosing spondylitis, anticardiolipin syndrome, autoimmune adrenalitis, autoimmune neutropenia, autoimmune oophoritis, autoimmune orchitis, autoimmune polyendocrine syndrome type 2, autoimmune sclerosing pancreatitis, Balanatis xerotica obliterans, Behcet's disease, benign recurrent meningitis, Calcinosis-Raynaud's sclerodactyly-telangiectasia syndrome, Caplan's disease, Churg-Strauss syndrome, cicatricial pemphigoid, Degos' disease, dermatitis herpetiformis, discoid lupus erythematosus, Dressler's syndrome, Eaton-Lambert syndrome, eosinophilic fasciitis, eosinophilic pustular folliculitis, epidermolysis bullosa acquisita, Evans syndrome, cryptogenic fibrosing alveolitis, Henoch-Schönlein purpura, Hughes-Stovin syndrome, hypertrophic pulmonary osteo-arthropathy, autoimmune hypoparathyroidism, inclusion body myositis, inflammatory bowel disease, insulin antibodies, insulin receptor antibodies, juvenile chronic arthritis, Kawasaki disease, linear IgA disease, lymphocytic mastisis, microscopic polyangiitis, Mikulicz's syndrome, Miller-Fisher syndrome, morphoea, acquired neuromyotonia, oculovestibuloauditory syndrome, paraneoplastic pemphigus, paroxysmal cold hemoglobinuria, partial lipodystrophy, polyarteritis nodosa, polychondritis, polymyalgia rheumatica, polyradiculoneuropathy, postpartum thyroiditis, primary biliary cirrhosis, primary sclerosing cholangitis, pyoderma gangrenosum, rhizomelic pseudopolyarthritis, sarcoidosis, Sicca syndrome, Sneddon-Wilkinson disease, Still's Disease, Susac's syndrome, sympathetic ophthalmitis, systemic sclerosis, Takayasu's arteritis, temporal arteritis, thrombangiitis obliterans, ulcerative colitis, vitiligo, Vogt-Koyanagi-Harada syndrome, Wegener's granulomatosis, rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Graves' disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, insulin-dependent diabetes mellitus, graft versus host disease, uveitis, rheumatic fever, Guillain-Barre syndrome, psoriasis, and autoimmune hepatitis.

In other embodiments, the adverse immune response results from exposure to allergens. The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

The symptoms of the allergic reaction vary, depending on the location within the body where the IgE reacts with the antigen. If the reaction occurs along the respiratory epithelium the symptoms are sneezing, coughing and asthmatic reactions. If the interaction occurs in the digestive tract, as in the case of food allergies, abdominal pain and diarrhea are common. Systematic reactions, for example following a bee sting, can be severe and often life threatening.

Delayed type hypersensitivity, also known as type IV allergy reaction is an allergic reaction characterized by a delay period of at least 12 hours from invasion of the antigen into the allergic subject until appearance of the inflammatory or immune reaction. The T lymphocytes (sensitized T lymphocytes) of individuals in an allergic condition react with the antigen, triggering the T lymphocytes to release lymphokines which function as inflammation mediators, and the biological activity of these lymphokines, together with the direct and indirect effects of locally appearing lymphocytes and other inflammatory immune cells, give rise to the type IV allergy reaction. Delayed allergy reactions include tuberculin type reaction, homograft rejection reaction, cell-dependent type protective reaction, contact dermatitis hypersensitivity reaction, and the like, which are known to be most strongly suppressed by steroidal agents. Consequently, steroidal agents are effective against diseases which are caused by delayed allergy reactions. Long-term use of steroidal agents at concentrations currently being used can, however, lead to the serious side-effect known as steroid dependence. The methods of the invention solve some of these problems, by providing for lower and fewer doses to be administered Allergic conditions or diseases in humans include but are not limited to eczema, allergic rhinitis or coryza, hay fever, conjunctivitis, bronchial or allergic asthma, urticaria (hives) and food allergies; atopic dermatitis; anaphylaxis; drug allergy; angioedema; and allergic conjunctivitis. Allergic diseases in dogs include but are not limited to seasonal dermatitis; perennial dermatitis; rhinitis: conjunctivitis; allergic asthma; and drug reactions. Allergic diseases in cats include but are not limited to dermatitis and respiratory disorders; and food allergens. Allergic diseases in horses include but are not limited to respiratory disorders such as “heaves” and dermatitis. Allergic diseases in non-human primates include but are not limited to allergic asthma and allergic dermatitis.

Immediate immune hypersensitivity (or anaphylactic response) is a form of allergic reaction which develops very quickly, i.e. within seconds or minutes of exposure of the patient to the causative allergen, and it is mediated by IgE antibodies made by B lymphocytes. In nonallergic patients, there is no IgE antibody of clinical relevance; but, in a person suffering with allergic diseases, IgE antibody mediates immediate hypersensitivity by sensitizing mast cells which are abundant in the skin, lymphoid organs, in the membranes of the eye, nose and mouth, and in the respiratory tract and intestines.

An “allergen” as used herein is a molecule capable of provoking an immune response characterized by production of IgE. Thus, in the context of this invention, the term allergen means a specific type of antigen which can trigger an allergic response which is mediated by IgE antibody. Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many allergens, however, are protein or polypeptide in nature, as proteins and polypeptides are generally more antigenic than carbohydrates or fats. Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenie or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Arteinisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chainaecyparis obtusa); Periplaneta (e.g. Periplaneta americana); Agropyron (e.g. Agropyron repens); Secale (e.g. Secale cereale); Triticum (e.g. Triticun aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In other embodiments, the adverse immune response results from exposure to infectious pathogens. Pathogens include, for example, viruses, bacteria, parasites, and fungi.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthoinyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals.

Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponena pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlamydia trachonzatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishnania spp., Leishmania braziliensis, Leishmania donovani, Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

The invention will be more fully understood by reference to the following examples. These examples, however, are merely intended to illustrate the embodiments of the invention and are not to be construed to limit the scope of the invention.

### EXAMPLES

### Example 1

### GMCSF Enhances FDG Uptake within Infected Lesions

In an effort to improve the detection of infectious foci, it was tested whether immune modulation with GMCSF, by enhancing macrophage metabolism, increases FDG uptake by immune cells associated with local candida infection, thereby enhancing detection by positron emission tomography (PET) imaging.

Candida albicans was suspended in saline to achieve a concentration of 2.5×10⁹ yeast cells/ml. Three mice (C57/BL, Jackson Laboratories, Bar Harbor, Me.) were injected intramuscularly in the left thigh with 5×10⁸ Candida albicans cells suspended in 0.2 ml of saline. The right thigh was injected with 0.2 ml of saline as a control.

Imaging of the animals was performed on days 5 and 8 after Candida inoculation. On each occasion, 18-Fluorodeoxyglucose (FDG) was administered via tail vein (2 mCi/Kg), and tomographic imaging was performed 3 hours later using a small animal positron emission tomography (PET) system, (CTI Concorde, Knoxyille, Tenn.). GMCSF was administered intramuscularly immediately after the first PET images were obtained (3 days prior to the second PET imaging).

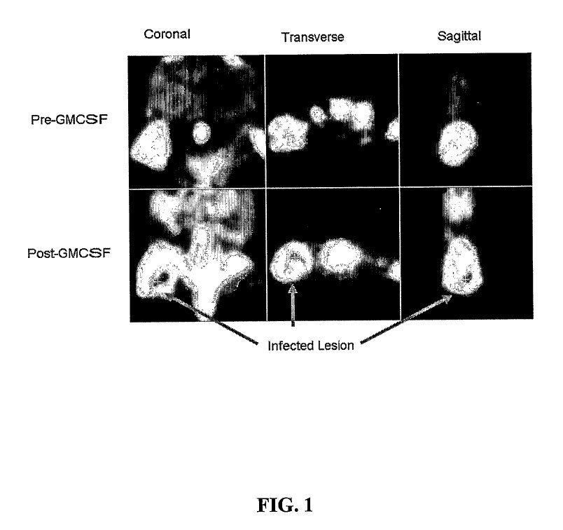

Prior to the administration of GMCSF, FDG uptake was increased within the infected lesion in all animals. Following the administration of GMCSF, FDG uptake within the infected lesion increased further in all animals (7070 nCi/cc vs. 8150 nCi/cc, pre vs. post-GMCSF, respectively, p<0.05, FIG. 1). These results indicate that GMCSF enhances FDG uptake within infected lesions. Accordingly, GMCSF administration prior to PET imaging may enhance the ability to detect infections.

Those skilled in the art will recognize, or be able to ascertain using no more than routine experimentation, many equivalents to the specific embodiments of the invention described herein. Such equivalents are intended to be encompassed by the following claims. All references disclosed herein are incorporated by reference in their entirety.

### REFERENCES

Akhlynina, T. V., Rosenkranz, A. A., Jans, D. A., and Sobolev, A. S.: “Insulin-mediated intracellular targeting enhances the photodynamic activity of chlorin e6” 1995. Cancer Res. 55(5): 1014-9.
Ashcroft, G. S., Lei, K., Jin, W., Longenecker, G., Kulkarni, A. B., Greenwell-Wild, T., Hale-Donze, H., McGrady, G., Song, X. Y., Wahl, S. M.: “Secretory leukocyte protease inhibitor mediates non-redundant functions necessary for normal wound healing” 2000 Nat. Med. 6(10): 1147-53.
Ausubel, F. M., Brent, R., Kingston, R., Moore, D., Seidman, J., Struhl, K. 1997. Current Protocols in Molecular Biology. John Wiley and Sons, New York, N.Y.
Aveline, B., Hasan, T., and Redmond, R. W.: “Photophysical and photosensitizing properties of benzoporphyrin derivative monoacid ring A (BPD-MA)” 1994. Photochem. Photobiol. 59(3): 328-35.
Bachor, R., Shea, C. R., Gillies, R., and Hasan, T.: “Photosensitized destruction of human bladder carcinoma cells treated with chlorin e6-conjugated microspheres” 1991. Proc. Natl. Acad. Sci. U.S.A. 88(4): 1580-4.
Balkwill, F. and Mantovani, A.: “Inflammation and cancer: back to Virchow?” 2001. Lancet 357: 539-545.
Basu, S., Mukhopadhyay, B., Basu, S. K., and Mukhopadhyay, A.: “Enhanced intracellular delivery of doxorubicin by scavenger receptor-mediated endocytosis for preferential killing of histiocytic lymphoma cells in culture” 1994. FEBS Lett. 342(3): 249-54.
Boehncke, W. H., and Schon, M. P.: “Interfering with leukocyte rolling—a promising therapeutic approach in inflammatory skin disorders?” 2003. Trends Pharm. Sci. 24: 49-52.
Coligan, J. E., Kruisbeek, A. M., Margulies, D. H., Shevach, E. M., Strober, W. 1991. Current Protocols in Immunology. John Wiley and Sons, New York, N.Y.
Corry, D. B., Rishi, K., Kanellis, J., Kiss, A., Song, L. Z., Xu, J., Feng, L., Werb, Z., Kheradmand, F.: “Decreased allergic lung inflammatory cell egression and increased susceptibility to asphyxiation in MMP2-deficiency” 2002 Nat. immunol. 3(4): 347-53.
Coussens, L. M., Werb, Z.: “Inflammation and cancer” 2002 Nature 420: 860-7.
Danpure, H. J., and Osman, S.: “Cell labelling and cell damage with indium-111 acetylacetonean alternative to indium 111 oxine” 1981. Br. J. Radiol. 54(643): 597-601.
Danpure, H. J., Osman, S., and Brady, F.: “The labelling of blood cells in plasma with ¹¹¹In-tropolonate” 1982. Br. J. Radiol. 55(651): 247-9.
Del Governatore, M., Hamblin, M. R., Piccinini, E. E., Ugolini, G., and Hasan, T.: “Targeted photodestruction of human colon cancer cells using charged 17.1A chlorin e6 immunoconjugates” 2000. Br. J. Cancer 82(1): 56-64.
Diomede, L. Albani, D., Sottocorno, M., Donati, M. B., Bianchi, M., Fruscella, P., Salmona, M.: “In vivo anti-inflammatory effect of statins is mediated by nonsterol mevalonate products” 2001. Arterioscler. Thromb. Vasc. Biol. 21(8): 13 27-32.
Dvorak, H. F.: “Tumors: wounds that do not heal. Similarities between tumor stroma generation and wound healing.” 1986. N. Engl. J. Med. 315: 1650-1659.
Eckelman, W. C., and Richards, P.: “Technetium labeling of albumin” 1972. J. Nucl. Med. 13(2): 180-1.
Epinat, J. C. and Gilmore, T. D.: “Diverse agents act at multiple levels to inhibit Rel/NF-κB signal transduction pathway” 1999. Oncogene 18: 6896-6909.
Ernst, P. B. and Gold, B. D.: “The disease spectrum of Helicobacter pylori: the immunopathogenesis of gastroduodenal ulcer and gastric cancer” 2000. Ann. Rev. Microbiol. 54: 615-640.
Freeman, M. W.: “Scavenger receptors in atherosclerosis” 1997. Curr. Opin. Hematol. 4(1): 41-7.
Freshney, R. I., 1987. Animal Cell Culture: A Practical Approach. John Wiley and Sons, New York, N.Y.
Funk, C. D.: “Prostaglandins and leukotrienes: advances in eicosanoid biology” 2001. Science 294(5548): 1871-5.
Gait, M. J. 1984. Oligonucleotide Synthesis: A Practical Approach. IRL Press, Oxford, United Kingdom.
Gennaro, A. R., Gennaro, A. L. Remington: The Science and Practice of Pharmacy, 17^{th} Edition, Mack Publishing Company, Easton, Pa.
Gijsens, A., and De Witte, P.: “Photocytotoxic action of EGF-PVA-Sn(IV) chlorin e6 and EGF-dextran-Sn(IV)chlorin e6 intenializable conjugates on A431 cells” 1998. Int. J. Oncol. 13(6): 1171-7.
Goodman Gilman, A., Hardman, J. G., Limbird, L. E., Molinoff, P. B., Ruddon, R. W. (eds) Goodman & Gilman's The Pharmacological Basis of Therapeutics 9^{th} Edition. (1996) McGraw-Hill, New York.
Grobmyer, S. R., Barie, P. S., Nathan, C. F., Fuortes, M., Lin, E., Lowry, S. F., Wright, C. D., Weyant, M. J., Hydro, L., Reeves, F., Shiloh, M. U., Ding, A.: “Secretory leukocyte protease inhibitor, an inhibitor of neutrophil activation, is elevated in serum in human sepsis and experimental endotoxemia” 2000 Crit. Care Med. 28(5): 1276-82.
Gunter, K. P., Lukens, J. N., Clanton, J. A., Morris, P. J., Janco, R. L., and English, D.: “Neutrophil labeling with indium-111: tropolone vs. oxine”. 1983. Radiology 149(2): 563-6.
Haberland, M. E., and Fogelman, A. M.: “Scavenger receptor-mediated recognition of maleyl bovine plasma albumin and the demaleylated protein in human monocyte macrophages” 1985. Proc. Natl. Acad. Sci. U.S.A. 82(9): 2693-7.
Hamblin, M. R., and Newman, E. L.: “Photosensitizer targeting in photodynamic therapy. I. Conjugates of haematoporphyrin with albumin and transferrin” 1994. J Photochem. Photobiol. B. 26(1): 45-56.
Hamblin, M. R., and Newman, E. L.: “Photosensitizer targeting in photodynamic therapy. II. Conjugates of haematoporphyrin with serum lipoproteins” 1994. J. Photochem. Photobiol. B. 26(2): 147-57.
Hamblin, M. R., Rajadhyaksha, M., Momma, T., Soukos, N. S., and Hasan, T.: “In vivo fluorescence imaging of the transport of charged chlorin e6 conjugates in a rat orthotopic prostate tumour” 1999. Br. J. Cancer 81(2): 261-8.
Hamblin, M. R., Del Govematore, M., Rizvi, I., and Hasan, T.: “Biodistribution of charged 17.1A photoimmunoconjugates in a murine model of hepatic metastasis of colorectal cancer”. 2000. Br. J. Cancer 83(11): 1544-51.
Hamblin, M. R., Miller, J. L., and Ortel, B.: “Scavenger-receptor targeted photodynamic therapy” 2000. Photochem. Photobiol. 72(4): 533-40.
Hasan, T. (1992) In: B. Henderson and T. Dougherty (eds.), Photodynamic Therapy: Basic Principles and Clinical Applications. Pp. 187-200: Marcel Dekker.
Harlow, E. and Lane, D. 1988. Antibodies: A Laboratory Manual. Cold Spring Harbor Laboratory, New York, N.Y.
Heitner, T., Moor, A., Garrison, J. L., Marks, C., Hasan, T., and Marks, J. D.: “Selection of cell binding and internalizing epidermal growth factor receptor antibodies from a phage display library” 2001. J. Immunol. Methods 248(1-2): 17-30.
Hodge-Dufour, J., Marino, M. W., Horton, M. R., Jungbluth, A., Burdick, M. D., Strieter, R. M., Noble, P. W., Hunter, C. A.: “Pure E Inhibition of interferon gamma induced interleukin 12 production: a potential mechanism for the anti-inflammatory activities of tumor necrosis factor” 1998 Proc. Natl. Acad. Sci. USA 95(23): 13806-11.
Hoffman, E. J., Huang, S. C., Phelps, M. E., Kuhl, D. E. “Quantitation in positron emission computed tomography: 4. Effect of accidental coincidences” 1981. J. Comput. Assist. Tomogr. 5: 391-400.
Hoffman, E. J., Phelps, M. E. “Positron emission tomography: principles and quantitation”. In: Phelps, M., Mazziotta, J., Schelbert, H., eds. Positron emission tomography and autoradiograply: principles and applications for the brain and heart. New York: Raven Press; 1986: 237-286.
Holgate, S. T., Peters-Golden, M., Paneffieri, R. A., Henderson W. R. Jr.: “Roles of cysteinyl leukotrienes in airway inflammation, smooth muscle function, and remodeling” 2003. J. Allergy Clin. Immunol. 111: S18-S34.
Jander, S., Sitzer, M., Schumann, R., Schroeter, M., Siebler, M., Steinmetz, H., and Stoll, C.: “Inflammation in high-grade carotid stenosis: a possible role for macrophages and T cells in plaque destabilization” 1998. Stroke 29(8): 1625-30.
Jaszczak, R. J. “SPECT: state-of-the-art scanners and reconstruction strategies”. In: Diksic, M., Reba, R. C., eds. Radiopharinaceuticals and brain pathology studied with PET and SPECT. Boca Raton: CRC Press; 1991: 93-118.
Jin, F. Y., Nathan, C., Radzioch, D., Ding, A. “Secretory leukocyte protease inhibitor: a macrophage product induced by and antagonistic to bacterial lipopolysaccharide” 1997 Cell 88(3): 417-26.
Keane, J., Gershon, S., Wise, R. P., Mirabile-Levens, E., Kasznica, J., Schwieterman, W. D., Siegel, J. N., Braun, M. M.: “Tuberculosis associated with infliximab, a tumor necrosis factor alpha-neutralizing agent” 2001. N. Engl. J. Med. 345(15): 1098-1104.
Kim, Y. J., Borsig, L., Varki, N. M., Varki, A.: “P-selectin deficiency attenuates tumor growth and metastasis.” 1998 Proc. Natl. Acad. Sci. USA 95: 9325-9330.
Krieger, M.: “Molecular flypaper and atherosclerosis: structure of the macrophage scavenger receptor” 1992. Trends Biochem. Sci. 17(4): 141-6. Krinick, N. L., Sun, Y., Joyner, D., Spikes, J. D., Straight, R. C., and Kopecek, J. A.: “Polymeric drug delivery system for the simultaneous delivery of drugs activatable by enzymes and/or light” 1994. J. Biomater. Sci. Polym. Ed. 5(4): 303-24.
Kuby, J. (1998) In: Immunology 3^{rd} Edition W.H. Freeman and Company, New York
Kuper, H., Adami, H. O., and Trichopoulos, D.: “Infections as a major preventable cause of human cancer.” 2000. J. Intern. Med. 248: 171-183.
Lee, D. M., Friend, D. S., Gurish, M. F., Benoist, C., Mathis, D., Brenner, M. B. “Mast cells: a cellular link between autoantibodies and inflammatory arthritis” 2002. Science 297: 1689-92.
Levy, B. D., Clish, C. B., Schmidt, B., Gronert, K., Serhan, C. N.: “Lipid mediator class switching during acute inflammation: signals in resolution” 2001 Nat. Immunol. 2(7): 612-9.
Levy, J. G.: “Photosensitizers in photodynamic therapy”. 1994. Semin. Oncol. 21(6 Suppl 15): 4-10.
Li, Q., and Verma, I. M.: “NF-κB regulation in the immune system” 2002. Nat. Rev. Immunol. 2: 725-34.
Links, J. M. “Physics and instrumentation of positron emission tomography”. In: Frost, J. J., Wagner, H. N., eds. Quantitative imaging: neuroreceptors, neurotransmitters, and enzymes. New York: Raven Press; 1990: 37-50.
Maeda, H. and Akaike, T. “Nitric oxide and oxygen radicals in infection, inflammation and cancer” 1998. Biochemistry 63: 854-865.
McAfee, J. G., and Thakur, M. L.: “Survey of radioactive agents for in vitro labeling of phagocytic leukocytes. I. Soluble agents” 1976. J. Nucl. Med. 17(6): 480-7
Molpus, K. L., Hamblin, M. R., Rizvi, I., and Hasan, T.: “Intraperitoneal photoimmunotherapy of ovarian carcinoma xenografts in nude mice using charged photoimmunoconjugates” 2000. Gynecol. Oncol. 76(3): 397-404.
Morgan, J. G., Pereira, H. A., Sukiennicki, T., Spitznagel, J. K., Larrick, J. V. “Human neutrophil granule cationic protein CAP37 is a specific macrophage chemotaxin that shares homology with inflammatory proteinases.” 1991. Adv. Exp. Med. Biol. 305: 89-96.
Mukhopadhyay, A., Mukhopadhyay, B., Srivastava, R. K., and Basu, S. K.: “Scavenger-receptor-mediated delivery of daunomycin elicits selective toxicity towards neoplastic cells of macrophage lineage”. 1992. Biochem. J. 284(Pt 1): 237-41.
Muller, A. Homey, B., Soto, H., Ge, N., Catron, D., Buchanan, M. E., McClanahan, T., Murphy, E., Yuan, W., Wagner, S. N., Barrera, J. L., Mohar, A., Verastegui, E., Zlotnik, A.: “Involvement of chemokine receptors in breast cancer metastasis” 2001 Nature 410(6824): 50-6.
Mullis, K. B., 1994. PCR: The Polymerase Chain Reaction. Birkhauser Press, Cambridge, Mass.
Nathan, C. F. “Neutrophil activation on biological surfaces. Massive secretion of hydrogen peroxide in response to products of macrophages and lymphocytes” 1987. J. Clin. Invest. 80(6): 1550-60.
Nathan, C., “Points of control in inflammation” 2002. Nature 420: 846-52.
Nathan, C., Xie, Q. W., Halbwachs-Mecarelli, L., Jin, W. W. “Albumin inhibits neutrophil spreading and hydrogen peroxide release by blocking the shedding of CD43 (sialophorin, leukosialin)” 1993 J. Cell Biol. 122: 243-56.
Niwa, S., Totsuka, T., Hayashi, S.: “Inhibitory effect of fluvastatin, an HMG-CoA reductase inhibitor, on the expression of adhesion molecules on human monocyte cell line” 1996. Int. J. Immunopharmacol. 18(11): 669-75.
Parrish, J. A., 1978. Optical properties of the skin and eyes. Plenum Press, New York, N.Y.
Pelaia, G., Vatrella, A., Cuda, G., Maselli, R., Marsico, S. A.: “Molecular mechanisms of corticosteroid actions in chronic inflammatory airway diseases” 2003. Life Sci. 72(14): 1549-61.
Peters, A. M., Saverymuttu, S. H., Reavy, H. J., Danpure, H. J., Osman, S., and Lavender. J. P.: “Imaging of inflammation with indium-111 tropolonate labeled leukocytes” 1983. J. Nucl. Med. 24: 39-44.
Peters, A. M., Danpure, H. J., Osman, S., Hawker, R. J., Henderson, B. L., Hodgson, H. J., Kelly, J. D., Neirinckx. R. D., and Lavender, J. P.: “Clinical experience with 99 mTc-hexamethylpropylene-amineoxime for labelling leucocytes and imaging inflammation” 1986. Lancet 2(8513): 946-9.
Phelps, M. E., Hoffman, E. J., Huang, S. C., Ter-Pogossian, M. M. “Effect of positron range on spatial resolution”. 1975. J Nucl Med 16: 649-652.
Polo, L., Segalla, A., Jori, G., Bocchiotti, G., Vema, G., Franceschini, R., Mosca, R., and De Filippi, P. G.: “Liposome-delivered ¹³¹I-labelled Zn(II)-phthalocyanine as a radiodiagnostic agent for tumours”. 1996. Cancer Lett. 109(1-2): 57-61.
Richmond, A. and Thomas, H.: “Purification of melanoma growth stimulatory activity” 1986. J. Cell. Physiol. 129: 375-384.
Richter, A. M., Kelly, B., Chow, J., Liu, D. J., Towers, G. H., Dolphin, D., Levy, J. G.: “Preliminary studies on a more effective phototoxic agent than hematoporphyrin” 1987. J. Natl. Cancer Inst. 79(6): 1327-32.
Robbiani, D. F., Finch, R. A., Jager, D., Muller, W. A., Sartorelli, A. C., Randolph, G. J. “The leukotriene C(4) transporter MRP1 regulates CCL19 (MIP-3beta, ELC)-dependent mobilization of dendritic cells to lymph nodes” 2000 Cell 103: 757-68.
Romano, M., Diomede, L., Sironi, M., Massimiliano, L., Soffocorno, M., Polentaruffi, N., Guglielmotti, A., Albani, D., Bruno, A., Fruscella, P., Salmona, M., Vecchi, A., Pinza, M., Mantovani, A.: “Inhibition of monocyte chemotactic protein-1 synthesis by statins”. 2000 Lab. Invest. 80(7): 1095-1100.
Sambrook, J. 1989. Molecular Cloning: A Laboratory Manual, second edition, Cold Spring Harbor Laboratory Press, New York, N.Y.
Schmidt-Erfurth, U., Diddens, H., Birngruber, R., and Hasan, T.: “Photodynamic targeting of human retinoblastoma cells using covalent low-density lipoprotein conjugates”. Br. J. Cancer 75(1): 54-61.
Sorenson, J. A., and Phelps, M. E. Physics in nuclear medicine, 2nd ed. Philadelphia: W.B. Saunders; 1987.
Sperduto, P. W., DeLaney, T. F., Thomas, G., Smith, P., Dachowski, L. J, Russo, A., Bonner, R., and Glatstein, E.: “Photodynamic therapy for chest wall recurrence in breast cancer” 1991. Int. Radiat. Oncol. Biol. Phys. 21(2): 441-6.
Srivastava, S. C., and Chervu, L. R.: “Radionuclide-labeled red blood cells: current status and future prospects” 1984. Semin. Nucl. Med. 14(2): 68-82.
Svaasand, L. O., and Ellingsen, R.: “Optical properties of human brain” 1983. Photochem. Photobiol. 38(3): 293-9.
Takata, K., Horiuchi, S., and Morino, Y.: “Scavenger receptor-mediated recognition of maleylated albumin and its relation to subsequent endocytic degradation” 1989. Biochim Biophys Acta 984(3): 273-80.
Thakur, M. L., Seifert, C. L., Madsen, M. T., McKenney, S. M., Desai, A G., Park, C. H.:
“Neutrophil labeling: problems and pitfalls” 1984. Semin. Nucl. Med. 14(2): 107-17.
Thakur, M. L., McKenney, S. L., and Park, C. H.: “Evaluation of indium-111-2-mercaptopyridine-N-oxide for labeling leukocytes in plasma: a kit preparation” 1985. J. Nucl. Med. 26: 518-523.
Tsimikas, S., Palinski, W., Halpern, S. E., Yeung, D. W., Curtiss, L. K., and Witztum, J. L.: “Radiolabeled MDA2, an oxidation-specific, monoclonal antibody, identifies native atherosclerotic lesions in vivo” 1999. J. Nucl. Cardiol. 6(1 Pt 1): 41-53
Vicari, A. P. and Caux, C. “Chemokines in cancer.” 2002. Cytokine Growth Factor Rev. 13: 143-154.
Wahl, L. M. and Kleinman, H. K. “Tumor-associated macrophages as targets for cancer therapy” 1998. J. Natl. Cancer Inst. 90: 1583-1584.
Walther, M. M., Delaney, T. F., Smith, P. D., Friauf, W. S., Thomas, G. F-, Shawker, T. H., Vargas, M. P., Choyke, P. L., Linehan, W. M., Abraham, E. H., Okunieff, P. G., and Glatstein, E.: “Phase I trial of photodynamic therapy in the treatment of recurrent superficial transitional cell carcinoma of the bladder” 1997. Urology 50(2): 199-206.
Weir, K. 1996. Handbook of Experimental Immunology Black-well Science, Inc. Boston, Mass.
Weiss, S. J. “Tissue destruction by neutrophils” 1989. N. Engl. J. Med. 320: 365-76.
Weitz-Schmidt, G.: “Statins as anti-inflammatory agents” 2002. Trends Pharm. Sci. 23(10): 482-6.
Welch, M. J., Thakur, M. L., Coleman, R. E., Patel, M., Siegel, B. A., and Ter-Pogossian, M.: “Gallium-68 labeled red cells and platelets: new agents for positron tomography” 1977. J. Nucl. Med. 18: 558-562.
Yamanishi, Y. Boyle, D. L., Rosengren, S., Green, D. R., Zvaifler, N. J., Firestein, G. S.: “Regional analysis of p53 mutations in rheumatoid arthritis synovium” 2002. Proc. Natl. Acad. Sci. USA 99(15): 10025-30.
Yang, D., Chertov, O., Bykovskaia, S. N., Chen, Q., Buffo, M. J., Shogan, J., Anderson, M., Schroder, J. M., Wang, J. M., Howard, O. M., Oppenheim, J. J. “Beta-defensins: linking innate and adaptive immunity through dendritic and T cell CCR6” 1999. Science 286: 525-8.
Yoshida, M. Sawada, T., Ishii, H., Gerszten, R. E., Rosenzweig, A., Gimbrone, M. A. Jr, Yasukochi, Y., Numano, F.: “HMG-CoA reductase inhibitor modulates monocyte-endothelial cell interaction under physiological flow conditions in vitro: involvement of Rho GTPasedependent mechanism” 2001. Arterioscler. Thromb. Vasc. Biol. 21(7): 1165-71.
Zhang, J., Nakayama, J., Ohyama, C., Suzuki, M., Suzuki, A., Fukuda, M., Fukuda, M. N.: “Sialyl Lewis X-dependent lung colonization of B16 melanoma cells through a selectin-like endothelial receptor distinct from E- or P-selectin” 2002 Cancer Res. 62(15): 4194-8.
Zhu, J., Nathan, C., Jin, W., Sim, D., Ashcroft, G. S., Wahl, S. M., Lacomis, L., ErdjumentBromage, H., Tempst, P., Wright, C. D., Ding, A.: “Conversion of proepithelin to epithelins: roles of SLPI and elastase in host defense and wound repair” 2002 Cell 111(6): 867-78.

1. A method of identifying inflamed tissue in a subject, said method comprising the steps of:
a) administering a diagnostic agent;
b) administering an immune modulator that increases localization of the diagnostic composition to inflammatory cells of the inflamed tissue;
c) detecting the diagnostic agent; and
d) identifying the inflamed tissue in the subject.

2. The method of claim 1, wherein the inflammatory cells are selected from the group consisting of smooth muscle cells, dendritic cells, follicular dendritic cells, Langerhans cells, interstitial, interdigitating, blood, and veiled dendritic cells, leukocytes, natural killer cells, lymphocytes, monocytes, macrophages, alveolar macrophages, microglia, mesangial cells, histiocytes, Kupffer cells, foam cells, mast cells, endothelial cells, megakaryocytes, platelets, erythrocytes and polymorphonuclear cells.

3. The method of claim 2, wherein the lymphocytes are B-lymphocytes or T-lymphocytes.

4. The method of claim 2, wherein the polymorphonuclear cells are granulocytes, basophils, eosinophils or neutrophils.

5. The method of claim 1, wherein the immune modulator is selected from the group consisting of colony stimulating factors, interleukins, interferons, chemokines, chemoattractants, growth factors, inhibitory factors, bacterially derived epitopes and signal transduction molecules.

6. The method of claim 1, wherein the immune modulator is selected from the group consisting of GM-CSF, M-CSF, G-CSF, interleukin-1 to -29, TNFα, formyl-methionine-leucine-phenylalanine (fMLP), lipopolysaccharide (LPS), phorbol 12-myristate-13-acetate, interferon α, interferon β, interferon γ, CD40, ligands of CD40, gp39, monocyte chemoattractant protein, basic fibroblast growth factor (bFGF), muramyl dipeptide, urokinase, regulated upon activation normally T-cell expressed and presumably secreted (RANTES), growth regulated oncogene, interferon-inducible T-cell alpha chemoattractant (1-TAC), monokine induced by gamma-interferon (MIG-1), leukemia inhibitory factor (LIF), oncostatin M, transforming growth factor β (TGF β), tissue inhibitor of matrix metalloproteinases (TIMP), macrophage chemotactic factor (MCF), and macrophage inflammatory protein.

7. The method of claim 1, wherein the diagnostic agent is selected from the group consisting of a photosensitizer, fluorescent marker and radiolabeled marker.

8. The method of claim 7, wherein the photosensitizer is motexafin lutetium.

9. The method of claim 7, wherein the photosensitizer is chlorinₑ₆.

10. The method of claim 7, wherein the photosensitizer is MV0633.

11. The method of claim 7, wherein the fluorescent marker is Fluorodeoxyglucose.

12. The method of claim 7, wherein the radiolabeled marker is a emitter.

13. The method of claim 12, wherein the β-emitter is selected from the group consisting of ¹³¹I, ¹²⁵I, ¹²³I, ^{99m}Tc, ¹⁸F, ⁶⁸Ga, ⁶⁷Ga, ⁷²As, ⁸⁹Zr, ⁶²Cu, ¹¹¹Cu, ²⁰³In, ¹⁹⁸Pb, ¹⁹⁸Hg, ⁹⁷Ru, ¹¹C, Re¹⁸⁸ and 201Tl.

14. The method according to claim 12, wherein the β-emitter is ¹⁸F-Fluorodeoxyglucose.

15. The method according to claim 12, wherein the β-emitter is ¹⁸⁸Re.

16. The method according to claim 12, wherein the diagnostic agent is a radiolabeled marker and the signal emitted by the radiolabeled marker is detected by positron emission tomography, magnetic resonance imaging, computer tomography, single photon emission computed tomography or a β-ray detector probe.

17. The method of claim 1, wherein the diagnostic agent is coupled to a molecular carrier.

18. The method of claim 17, wherein the molecular carrier targets the diagnostic agent to inflammatory cells selected from the group consisting of smooth muscle cells, dendritic cells, follicular dendritic cells, Langerhans cells, interstitial, interdigitating, blood, and veiled dendritic cells, leukocytes, natural killer cells, lymphocytes, monocytes, macrophages, alveolar macrophages, microglia, mesangial cells, histiocytes, Kupffer cells, foam cells, mast cells, endothelial cells, megakaryocytes, platelets, erythrocytes and polymorphonuclear cells.

19. The method of claim 18, wherein the lymphocytes are B-lymphocytes or T-lymphocytes.

20. The method of claim 18, wherein the polymorphonuclear cells are granulocytes, basophils, eosinophils or neutrophils.

21. The method of claim 17, wherein the molecular carrier is selected from the group consisting of serum proteins, receptor ligands, microspheres, liposomes, antibodies, growth factors, peptides, hormones and lipoproteins.

22. The method of claim 17, wherein the molecular carrier binds to a scavenger receptor.

23. The method of claim 22, wherein the molecular carrier is selected from the group consisting of maleylated albumin, daunorubicin, doxorubicin, oxidized low density lipoprotein, acetylated low density lipoprotein, oxidized high density lipoprotein, malondialdehyde treated proteins, formaldehyde treated albumin, glycated albumin, polyinosinic acid, glycated lipoproteins, dextran sulfate, anionic phospholipids, fucoidin, carrageenan, polyvinyl sulfate and monoclonal antibodies that recognize CD11b, CD11c, CD13, CD14, CD16a, CD32 or CD68.

24. The method of claim 23, wherein the anionic phospholipid is phosphatidyl serine.

25. The method of claim 17, where in the molecular carrier targets the diagnostic agent to a T-cell.

26. The method of claim 25, wherein the molecular carrier targets the diagnostic agent to a T cell biomolecule selected from the group consisting of IL-10, IL-10 receptor, monocyte inflammatory protein-1, monocyte inflammatory protein-1 receptor and transferrin.

27. The method of claim 25, where in the molecular carrier is selected from the group consisting of monoclonal antibodies that recognize CD1, CD2, CD3, CD4, CD5, CD6, CD7, CD8, CD25, CD28, CD44 and CD71 and transferrin.

28. The method of claim 17, where in the molecular carrier targets the diagnostic agent to lipids of the inflamed tissue.

29. The method of claim 28, wherein the molecular carrier comprises hydrophobic vehicles selected from the group consisting of liposomes, cremaphor EL, PEG/solvent mixtures, iodized castor oil, nanoparticles and micellar preparations.

30. The method of claim 29, wherein the liposomes contain cholesterol.

31. The method of claim 29, wherein the liposomes contain cardiolipin.

32. The method of claim 17, wherein the molecular carrier targets the diagnostic agent to macrophages.

33. The method of claim 32, wherein the molecular carrier targets the diagnostic agent to a macrophage biomolecule selected from the group consisting of For-Met-Leu-Phe, tenascin C, tissue factor, tissue inhibitor of MMP 1, tissue inhibitor of MMP 2, oxidized LDL receptor, heme oxygenase-1, human cartilage gp-39, IL-6, IL-6 receptor, IL-10, IL-10 receptor, lectin-like oxidized LDL-receptor, monocyte inflammatory protein-1, monocyte inflammatory protein-1 receptor and macrophage chemoattractant protein-1 receptor.

34. The method of claim 17, wherein the molecular carrier targets the diagnostic agent to foam cells.

35. The method of claim 17, wherein the molecular carrier that targets the diagnostic agent is a protease that degrades extracellular matrix.

36. The method of claim 35, wherein the protease is a metalloproteinase.

37. The method of claim 35, wherein the molecular carrier is a monoclonal antibody that binds to an epitope on a protease.

38. The method of claim 1, wherein the subject is a human.

39. The method of claim 1, wherein the inflamed tissue is infected.

40. The method of claim 1, wherein the inflamed tissue is transplanted tissue.

41. The method of claim 1, wherein the subject has an autoimmune disorder that produced the inflamed tissue.

42. A method for identifying inflamed tissue in a subject, said method comprising the steps of:
a) administering a diagnostic agent;
b) administering an immune modulator that increases localization of the diagnostic composition to inflammatory cells of the inflamed tissue;
c) comparing a signal emitted by the diagnostic agent in the inflamed tissue to a signal emitted by the diagnostic agent in another tissue; and
d) determining the location of the greater amount of signal to thereby identify the inflamed tissue in the subject.

43-82. (canceled)

83. A method for treating inflamed tissue in a subject in need thereof, said method comprising the steps of:
a) administering a therapeutic agent;
b) administering an immune modulator that increases localization of the therapeutic agent to inflammatory cells of the inflamed tissue, thereby treating the subject for inflamed tissue.

84-114. (canceled)

115. A method for identifying and treating inflamed tissue in a subject in need thereof with the use of photodynamic means, said method comprising the steps of:
a) administering at least one photosensitizer;
b) administering an immune modulator that increases localization of the photosensitizer to inflammatory cells of the inflamed tissue;
c) detecting a sufficient amount of the photosensitizer to thereby identify the inflamed tissue; and
d) irradiating the photosensitizer to produce a phototoxic species that destroys the inflammatory cells, thereby treating the subject for inflamed tissue.

116. The method of claim 1 further comprising obtaining the diagnostic agent and/or the immune modulator and/or the therapeutic agent and/or the photosensitizer.

117. A kit for identifying inflamed tissue comprising a diagnostic agent, an immune modulator and instructions for using the diagnostic agent and the immune modulator to identify inflamed tissue in accordance with the method of of claim 1.

118. (canceled)

119. A kit for treating inflamed tissue in a subject in need thereof comprising a therapeutic agent, an immune modulator and instructions for treating inflamed tissue using the therapeutic agent and the immune modulator in the subject in accordance with the method of claim 83.

120. A kit for detecting and treating inflamed tissue in a subject in need thereof comprising a photosensitizer, an immune modulator, and instructions for using the photosensitizer and the immune modulator to detect and treat inflamed tissue in a subject in accordance with the method of claim 115.

121. (canceled)
